(19) United States
(12) Patent Application Publication
(10) Pub. No.: US 2024/0050733 A1
(43) Pub. Date: Feb. 15, 2024

(54) SYSTEMS AND METHODS FOR ENHANCING NEUROSTRUCTURAL DEVELOPMENT

(71) Applicant: ElectroCore, Inc., Rockaway, NJ (US)

(72) Inventors: Bruce J. Simon, Santa Fe, NM (US); Joseph P. Errico, Palm Beach Gardens, FL (US)

(73) Assignee: ElectroCore, Inc., Rockaway, NJ (US)

(21) Appl. No.: 18/492,739

(22) Filed: Oct. 23, 2023

Related U.S. Application Data
(63) Continuation of application No. 17/844,368, filed on Jun. 20, 2022.

(51) Int. Cl. A61N 1/04; A61N 1/36; A61N 1/40

(52) U.S. Cl. CPC A61N 1/0456; A61N 1/36034; A61N 1/36053; A61N 1/40; A61M 2021/0022

(57) ABSTRACT

Systems and methods are provided for delivering energy impulses (and/or fields) to bodily tissues. In certain aspects, the systems and methods are particularly useful for temporarily or permanently improving intelligence, learning capacity, memory retention, recall, mood, alertness and/or sleep efficiency in human beings. In certain embodiments, the methods and devices enhance neurostructural development over a period of time by increasing neurogenesis, neuronal plasticity and/or neural connectivity efficiency, and/or by improving the chemical microenvironment of the evolving neural network. In some embodiments, these enhancements may provide temporary improvement to enable an individual to, for example, accomplish a particular task while under duress, sleep deprivation, stress, anxiety or the like. In other embodiments, these changes permanently enhance the intelligence, emotional stability, and overall brain health of the treated individual.

### CROSS REFERENCE TO RELATED APPLICATIONS

This patent application is a continuation of U.S. patent application Ser. No. 17/844,368, filed Jun. 21, 2022, the complete disclosure of which is incorporated by reference for all purposes as if copied and pasted herein.

This patent application is also related to the following commonly-assigned patents and patent applications: U.S. Nonprovisional application Ser. No. 16/229,299, filed Dec. 21, 2018, U.S. Nonprovisional application Ser. No. 17/491,313, filed Sep. 30, 2021, U.S. Nonprovisional application Ser. No. 17/002,347, filed Aug. 25, 2020, U.S. Nonprovisional application Ser. No. 16/838,953, filed Apr. 2, 2020, U.S. Nonprovisional application Ser. No. 17/471,962, filed Sep. 10, 2021, U.S. Pat. Nos. 11,229,790, 9,375,571, U.S. Nonprovisional application Ser. No. 13/858,114 filed 8 Apr. 2013, now U.S. Pat. No. 9,248,286 issued 2 Feb. 2016, U.S. Nonprovisional application Ser. No. 14/930,490 filed 2 Nov. 2015, now U.S. Pat. No. 10,507,325, U.S. Nonprovisional application Ser. No. 13/222,087 filed 31 Aug. 2011, now U.S. Pat. No. 9,174,066 issued 3 Nov. 2015, U.S. Nonprovisional application Ser. No. 13/183,765 filed 15 Jul. 2011, now U.S. Pat. No. 8,874,227 issued 28 Oct. 2014, U.S. Nonprovisional application Ser. No. 13/183,721 filed 15 Jul. 2011, now U.S. Pat. No. 8,676,324 issued 18 Mar. 2014, U.S. Nonprovisional application Ser. No. 13/109,250 filed 17 May 2011, now U.S. Pat. No. 8,676,330 issued 18 Mar. 2014, U.S. Nonprovisional application Ser. No. 13/075,746 filed 30 Mar. 2011, now U.S. Pat. No. 8,874,205 issued 28 Oct. 2014, U.S. Nonprovisional application Ser. No. 13/005,005 filed 12 Jan. 2011, now U.S. Pat. No. 8,868,177 issued 21 Oct. 2014, U.S. Nonprovisional application Ser. No. 12/964,050 filed 9 Dec. 2010, U.S. Nonprovisional application Ser. No. 12/859,568 filed 19 Aug. 2010, now U.S. Pat. No. 9,037,247 issued 19 May 2015, U.S. Nonprovisional application Ser. No. 12/612,177 filed 4 Nov. 2009, now U.S. Pat. No. 8,041,428 issued 18 Oct. 2011, U.S. Nonprovisional application Ser. No. 12/408,131 filed 20 Mar. 2009, now U.S. Pat. No. 8,812,112 issued 19 Aug. 2014, U.S. Nonprovisional application Ser. No. 15/149,406 filed 9 May 2016, U.S. Nonprovisional application Ser. No. 14/337,930 filed 22 Jul. 2014, now U.S. Pat. No. 9,333,347 issued 10 May 2016, U.S. Nonprovisional application Ser. No. 13/075,746 filed 30 Mar. 2011, now U.S. Pat. No. 8,874,205 issued 28 Oct. 2014, U.S. Nonprovisional application Ser. No. 12/964,050 filed 9 Dec. 2010, U.S. Nonprovisional application Ser. No. 12/859,568 filed 19 Aug. 2010, now U.S. Pat. No. 9,037,247 issued 19 May 2015, U.S. Nonprovisional application Ser. No. 14/462,605 filed 19 Aug. 2014, U.S. Nonprovisional application Ser. No. 13/005,005 filed 12 Jan. 2011, now U.S. Pat. No. 8,868,177 issued 21 Oct. 2014, U.S. Nonprovisional application Ser. No. 12/964,050 filed 9 Dec. 2010, U.S. Nonprovisional application Ser. No. 12/859,568 filed 19 Aug. 2010 now U.S. Pat. No. 9,037,247 issued 19 May 2015 and U.S. Nonprovisional application Ser. No. 12/408,131 filed 20 Mar. 2009 now U.S. Pat. No. 8,812,112 issued 19 Aug. 2014; all of which are hereby incorporated by reference in their entirety for all purposes as if copied and pasted herein.

### BACKGROUND

Over the past two decades, a significant volume of research in the fields of neuroimmunology and neuropsychoimmunology has demonstrated that microglia play a range of critical roles in the structural development of the central nervous system, beginning in utero, and extending through the decades of childhood and adolescence, and even impacting the maturation of the brain into early adulthood. Similar roles for microglia continue throughout life, including the pruning of synapses, formation of new neural connections, and generally managing the plasticity and excitability state of the brain that is necessary for learning by, the memory within, and the defense of the neural network. The latter requires microglia to take an aggressive posture, while the former are associated with a neuroprotective and/or neurotrophic state characterized by a highly ramified structure with long tendrils actually touching all of the neuronal and astrocytic surfaces, including axons and synapses in the vicinity.

More specifically, with respect to brain development, early phases of in utero and postnatal growth are characterized by rapid creation and integration of neurons into the brain's emerging structures. It is believed that microglia, which migrate into the brain by the end of the first week of pregnancy, actively guide the laying down of the tracts connecting white matter, for example the corpus collosum, and enable the high rate of connectivity throughout the brain. By age two years, the human brain has nearly 600 million neuronal connections per cubic millimeter. This high concentration of connections serves as the raw block of marble from which the sculpting of the more functional, but less connected brain of an adult will be carved. Microglia, in response to sensory, emotional, and cognitive inputs, prune connections that are weak and non-useful, while reinforcing and protecting synapses that are positive and necessary. By adulthood, the human brain typically has approximately 370 million connections per cubic millimeter, or a reduction of nearly 40%.

Dysfunction among microglia can lead to a number of disorders ranging from autism spectrum disorder, ASD, to schizophrenia, to Alzheimer's Disease and major depressive disorder. For example, it is believed that maternal or early childhood severe inflammatory events, which can include serious emotional trauma as well, can disrupt microglial function leading to a failure to prune efficiently, which can lead to ASD, or too much pruning which is associated with schizophrenia.

The ability of microglia to function in an optimally neurotrophic manner is dictated by the demands placed on them. Microglia are classically thought of as having two opposing states; one that is constructive and supportive of neural growth and enhanced functional capacity of the network, and the other that is designed to protect and defend the overall system from damage and can be cytotoxic and destructive. While these gross categorizations have been rightly criticized as being overly limiting in their appreciation of the myriad of tasks and substrates that microglia can assume, the morphological states of these immune cells, and the actions they take, do roughly correlate with the degree to which they are oriented toward a pro-inflammatory state versus an anti- or non-inflammatory one. Interestingly, the degree of inflammation, which can be acute or chronic, can be triggered by both direct physical injury (i.e., trauma, pathogens, and toxins) and more abstract damage (i.e., lack of sleep, prolonged emotional stress, and even pathological hyperexcitation). Not surprisingly, the quiescent, or neuroprotective state of the microglia is one in which the neurotrophic factors (e.g., BDNF) are expressed. It has been shown in multiple animal models that a neuroprotective microglia phenotype promotes neurogenesis by reducing the expression of proinflammatory cytokines such as TNF-α, and by increasing the expression of anti-inflammatory cytokines such as IL-1ra or the chemokine CX3CL1.

The expression of TNF-α in the central nervous system has broad effects, including, but not limited to, the disruption of the synthesis of multiple inhibitory neurotransmitters, alteration of expression levels of key transporter proteins associated with multiple neurotransmitters, triggering production of peptides that are associated with pain and vasoconstriction, and upregulating expression of receptors for neural excitation. As an example, the presence of TNF-α alters levels of serotonin through the upregulation in the expression of indolamine 2,3 dioxygenase (and through the HPA axis, tryptophan dioxygenase) both of which alter the favored biosynthetic pathway in the direction of kynurenine and suppresses the production of serotonin.

TNF-α also upregulates the expression of SERT (serotonin reuptake transporter) on the surface of astrocytes (the class of glial cells, distinct from microglia, that surround and support neurons and their synapses). By enhancing the expression of SERT, TNF-α effectively strips neurons of their ability to properly signal. Since serotonin is an inhibitory neurotransmitter critical to pain perception (serotonin can raise the threshold for pain perception through its role in descending inhibition), normal pain tolerance, mood, and the ability to concentrate are all disrupted. TNF-α also causes the upregulation of the production of ceramides, which in turn causes the synthesis and release of calcitonin gene-related peptide, or CGRP. CGRP is one of the most potent vasoconstrictors (released to choke off blood flow to a possible traumatic brain injury), and is implicated in migraine. TNF-α also causes the upregulation in expression of NMDA and AMPA receptors on neurons, and downregulating GABA_{A} receptors, which collectively has the effect of making the cell more prone to hyperexcitation (NMDA and AMPA receptors for the excitatory neurotransmitter glutamate), less sensitive to the inhibitory neurotransmitter GABA. Within the affected neuron, these changes cause the production of reactive oxygen species (ROS) which, upon release, block critical uptake of excess glutamate by astrocytes, thus further exacerbating the likelihood of hyperexcitability and enhancing the cell's susceptibility to excitotoxicity (neuronal death associated with activation of internal cell death signaling pathways).

What is needed, therefore, are therapies, treatments or protocols for temporarily and/or permanently improving brain function by enhancing neurostructural development in the brain of individuals, particularly in younger adults or children. It would be particularly desirable to provide protocols that enhance the ability of microglia in the central nervous system to support neural growth and the functional capacity of neural networks in the brain.

### SUMMARY

Systems and methods are provided for delivering energy impulses (and/or fields) to bodily tissues. In certain aspects, the systems and methods are particularly useful for temporarily or permanently improving intelligence, learning capacity, memory retention, recall, mood, alertness and/or sleep efficiency in human beings. In certain embodiments, the methods and devices enhance neurostructural development over a period of time by increasing neurogenesis, neuronal plasticity and/or neural connectivity efficiency, and/or by improving the chemical microenvironment of the evolving neural network. In some embodiments, these enhancements may provide temporary improvement to enable an individual to, for example, accomplish a particular task while under duress, sleep deprivation, stress, anxiety or the like. In other embodiments, these changes permanently enhance the intelligence, emotional stability, and overall brain health of the treated individual.

In one aspect, a method for modifying microglia in a human comprises applying an electrical impulse to a vagus nerve within the human. The electrical impulse is sufficient to modify a microglia in a central nervous system of the individual, such as in the brain. In particular, the electrical impulse is sufficient to alter, shift, move or otherwise change the microglia from a substantially pro-inflammatory state or orientation to a substantially non-inflammatory state or orientation.

The non-inflammatory state of microglia, also referred to as the quiescent, or neuroprotective state, is generally constructive and supportive of neural growth and enhanced functional capacity of the network. Altering the state of microglia from the inflammatory state to the non-inflammatory state promotes neurogenesis, increases connectivity between neurons and improves the overall environment of the neural network. by reducing the expression of proinflammatory cytokines. On the other hand, the inflammatory state of microglia functions to protect and defend the overall system from damage and can be cytotoxic and destructive. To enable suitable, balanced maintenance of brain homeostasis, microglia need to remain in the physiological and ramified (i.e., non-inflammatory) state.

In embodiments, the electrical impulse is sufficient to decrease expression of proinflammatory cytokines, such as TNF-α, Interleukin (IL)-1, IL-6, IL-8, IL-11 and other chemokines. In a preferred embodiment, the electrical impulse is sufficient to reduce the expression of TNF-α, which limits the disruption of the synthesis of multiple inhibitory neurotransmitters, alters the expression levels of key transporter proteins associated with multiple neurotransmitters, triggers the production of peptides that are associated with pain and vasoconstriction, and upregulates the expression of receptors for neural excitation.

In embodiments, the electrical impulse is sufficient to increase the expression of anti-inflammatory cytokines, such as IL-1ra or a CX3CL1 chemokine, (IL)-1 receptor antagonist, IL-4, IL-10, IL-11, and IL-13 and the like. In a preferred embodiment, the electrical impulse increases the expression of IL-1ram, CX3CL1 chemokines, brain-derived neurotrophic factors (BDNF).

In another aspect, a method for enhancing neurostructural development in an individual comprises applying an electrical impulse to a vagus nerve within the individual according to a stimulation protocol that includes at least two doses administered each day for a plurality of days. The doses each have a duration of about ninety seconds to about 3 minutes. The stimulation protocol is sufficient to increase an effectiveness of a neural network in a brain of the individual.

The effectiveness of the neural network may be increased through neurogenesis, or the creation of more neurons in the brain. Alternatively, or in addition, and depending on the timing thereof within the framework of development, the effectiveness may be enhanced by increasing a connectivity of neurons within the brain of the individual and/or increasing the effective pruning of connections, or enhancing a neuronal plasticity within the brain of the individual. Neuronal plasticity is generally defined as the ability of the brain to change its structure and/or function in response to previous experience. It is essential for the establishment and refinement of neural networks during development and the formation of memory traces, the acquisition of specific skills and the storage of information.

In certain embodiments, the effectiveness of the neural network is increased sufficiently to temporarily or permanently improve the intelligence, learning capacity, memory retention, recall, mood, alertness and/or sleep efficiency in the individual. These enhancements may provide temporary improvement to enable an individual to, for example, accomplish a particular task while under duress, sleep deprivation or the like. In other embodiments, these changes permanently enhance the intelligence, emotional stability, and overall brain health of the treated individual.

In another aspect, systems and methods are providing for increasing the neurostructural development of a child to permanently increase the intelligence, learning capacity and/or memory retention of the child as the child grows and develops. The child may, for example, have an age of less than 18 years old, less than 10 years old, less than 5 years old or even less than 2 years old. In a related aspect, the child may still be within the womb, and the modulation of a maternal inflammation state may be desirable.

In one embodiment, the electrical impulse is transmitted for at least 30 seconds and may be applied in a single dose for a time period of about 30 seconds and about 3 minutes, preferably about 90-150 seconds, or it may be applied in a series of doses each having a time period of about 30 seconds to about 3 minutes, preferably about 90-150 seconds in each dose. The series of doses may be applied every 5 to 30 minutes, every hour, 2-12 times per day, 4-6 times per day, or 2-4 times per day. In one embodiment, the doses are applied two times per day.

The doses may be applied every day, every other day, or once a week. The doses may be applied for a period of one month, three months, one year, three years, or longer. In one embodiment, two doses are applied every day for a period of three to five years.

In embodiments, the electrical impulse comprises pulses having a frequency of about 1 kHz to about 20 kHz. The electrical impulse may comprise bursts of pulses, with each burst having a frequency of about 1 to about 100 bursts per second and each pulse has a duration of about 50 to about 1000 microseconds in duration. The bursts each comprise about 2 to 20 pulses and the bursts are separated by an inter-burst period that comprises zero pulses.

In embodiments, the device further comprises a housing, such as a handheld device, that may be operated by the individual. The energy source is housed within the housing and the electrodes are attached to, or incorporated into, the housing.

The housing may contain the electronic components, signal generator and energy source that are used to generate the signals that drive electrical impulses through the electrodes. However, in other embodiments, the electronic components that generate the signals may be in a separate housing or device, such as a mobile device. Furthermore, other embodiments may contain a single electrode or more than two electrodes.

In other embodiments, the device comprises a patch having at least one adhesive surface for attachment to the outer skin surface of the neck of the individual. The electrodes are housed within the patch. The patch may further comprise a signal generator and an energy source for applying the electrical impulses through the electrodes to the vagus nerve. Alternatively, the patch may include a wireless receiver and associated electronics for wirelessly receiving the electrical impulse and/or the energy from the energy source.

The device may further comprise a controller coupled to the energy source and configured to transmit parameters for the stimulation protocol to the energy source. The controller and/or the energy source may be wirelessly coupled to the electrodes, or each other. Alternatively, the controller and the energy source may be housed within the patch or the handheld device.

In certain embodiments, the energy source is wirelessly coupled to the one or more electrodes. In other embodiments, the energy source is coupled to the electrodes directly with electrical connectors. In yet other embodiments, the energy source and the electrodes are housing within a handheld device that can be placed or attached against the outer surface of the individual's neck.

In one such embodiment, the electrodes are adhered to the outer skin surface of the individual's neck with a suitable adhesive. This allows the individual to be treated without direct intervention (i.e., holding a device or the electrodes against the individual's neck during stimulation). The system may further comprise an outer sheath or other wearable device, such as an insulating strip, a collar, or a garment, such as a turtleneck, a scarf or the like, that functions to adhere the electrodes to the neck of the individual. The electrodes may be housed within the wearable device, or positioned between the wearable device and the neck of the individual.

Various technologies for preventing, diagnosing, monitoring, ameliorating, or treating medical conditions, diseases, or disorders, such as replicating pathogens, are more completely described in the following detailed description, with reference to the drawings provided herewith, and in claims appended hereto. Other aspects, features, advantages, etc. will become apparent to one skilled in the art when the description is taken in conjunction with the accompanying drawings.

### INCORPORATION BY REFERENCE

Hereby, all issued patents, published patent applications, and non-patent publications that are mentioned in this specification are herein incorporated by reference in their entirety for all purposes as if copied and pasted herein, to the same extent as if each individual issued patent, published patent application, or non-patent publication were specifically and individually indicated to be incorporated by reference and copied and pasted herein.

### DESCRIPTION OF DRAWINGS

FIG. 1 illustrates a schematic view of one embodiment of a nerve modulating system;

FIG. 2A shows an embodiment of an electrical voltage/current profile for stimulating and/or modulating impulses that are applied to a nerve;

FIG. 2B illustrates an embodiment of a bursting electrical waveform for stimulating and/or modulating a nerve;

FIG. 2C illustrates an embodiment of two successive bursts of the waveform of FIG. 2B;

FIG. 3A is a perspective view of a stimulator;

FIG. 3B is a perspective view of the stimulator of FIG. 3A flipped upside down;

FIG. 3C is a perspective view of another embodiment of a stimulator with a cover for protecting the electrodes;

FIG. 3D is a perspective view of the stimulator of FIG. 3C with the cover positioned to expose the electrodes;

FIG. 4 is a perspective view of another embodiment of a stimulator;

FIG. 5 illustrates the stimulator of FIG. 4 when positioned to stimulate a vagus nerve in an individual's neck;

FIG. 6A is a front view of another embodiment of a stimulator;

FIG. 6B is a side view of the stimulator of FIG. 6A;

FIG. 7A is a front view of another embodiment of a stimulator;

FIG. 7B is a back view of the stimulator shown in FIG. 7A;

FIG. 7C is a side view of the stimulator shown in FIG. 7A;

FIG. 8 shows an expanded diagram of an embodiment of a control unit;

FIG. 9 illustrates an embodiment of an approximate position of a stimulator when used to stimulate a right vagus nerve in a neck of an adult individual;

FIG. 10 illustrates an embodiment of an approximate position of a stimulator when used to stimulate a right vagus nerve in a neck of a child who wears a collar to hold the stimulator;

FIG. 11 illustrates a system for modulating the vagus nerve;

FIG. 12 illustrates a patch stimulator device for attaching to a skin surface of an individual; and

FIG. 13A is a top view of an electrode array; and

FIG. 13B is an exploded side view of the electrode array of FIG. 13A.

### DETAILED DESCRIPTION

Methods and devices are provided for temporarily or permanently improving intelligence, learning capacity, memory retention, recall, mood, alertness and/or sleep efficiency in human beings. In certain embodiments, the methods and devices enhance neurostructural development over a period of time by increasing neurogenesis, neuronal plasticity, neural connectivity efficiency, and/or by improving the chemical microenvironment of the evolving neural network. These enhancements may provide temporary improvement to enable an individual to, for example, accomplish a particular task while under duress, sleep deprivation or the like. In other embodiments, these changes permanently enhance the intelligence, emotional stability, and overall brain health of the treated individual.

The methods and devices disclosed herein can be used to treat individuals that are generally considered healthy and/or they may be used to prevent, diagnose, monitor, ameliorate, or treat a medical condition, a disease, or a disorder of an individual, such as a mammal, such as an animal, such as a human, whether male or female, whether infant, child, adult, or elderly, or others.

In particular, the devices can transmit energy to, or in close proximity to, a selected nerve of the individual in order to temporarily stimulate, block and/or modulate electrophysiological signals in that nerve. In some embodiments, some electrodes applied to the skin of the individual generate currents within the tissue of the individual. This may enable production and application of the electrical impulses so as to interact with the signals of one or more nerves, in order to achieve the therapeutic result.

In some embodiments, methods and devices are specifically designed for the treatment of an individual by stimulation in or around a vagus nerve, with devices positioned non-invasively on or near an individual's neck. However, it will be recognized that some of the treatment paradigms can be used with a variety of different vagal nerve stimulators, including implantable and/or percutaneous stimulation devices.

In some embodiments, a time-varying magnetic field, originating and confined to the outside of an individual, generates an electromagnetic field and/or induces eddy currents within tissue of the individual. In some embodiments, electrodes applied to the skin of the individual generate currents within the tissue of the individual.

VNS was developed initially for the treatment of partial onset epilepsy and was subsequently developed for the treatment of depression and other disorders. The left vagus nerve is ordinarily stimulated at a location within the neck by first implanting an electrode about the vagus nerve during open neck surgery and by then connecting the electrode to an electrical stimulator circuit (a pulse generator). The pulse generator is ordinarily implanted subcutaneously within a pocket that is created at some distance from the electrode, which is usually in the left infraclavicular region of the chest. A lead is then tunneled subcutaneously to connect the electrode assembly and pulse generator. The individual's stimulation protocol is then programmed using a device (a programmer) that communicates with the pulse generator, with the objective of selecting electrical stimulation parameters that best treat the individual's condition, e.g., pulse frequency, stimulation amplitude, pulse width, etc.

Recently, minimally invasive electrical stimulators that transmit energy to nerves non-invasively or percutaneously have become more common. A medical procedure is defined as being non-invasive when no break in the skin (or other surface of the body, such as a wound bed) is created through use of the method, and when there is no contact with an internal body cavity beyond a body orifice (e.g., beyond the mouth or beyond the external auditory meatus of the ear). Such non-invasive procedures are distinguished from invasive procedures (including minimally invasive procedures) in that the invasive procedures insert a substance or device into or through the skin (or other surface of the body, such as a wound bed) or into an internal body cavity beyond a body orifice.

Non-invasive medical methods and devices provide a number of advantages relative to comparable invasive procedures. For example, the individual may be more psychologically prepared to experience a procedure that is non-invasive and may therefore be more cooperative, resulting in a better outcome. Non-invasive procedures may avoid damage of biological tissues, such as that due to bleeding, infection, skin or internal organ injury, blood vessel injury, and vein or lung blood clotting. Non-invasive procedures can be generally measurably painless and may be performed without some of the dangers and costs of surgery. They are ordinarily performed even without the need for local anesthesia. Less training may be required for use of non-invasive procedures by medical professionals. In view of the reduced risk ordinarily associated with non-invasive procedures, some such procedures may be suitable for use by the individual or family members at home or by first-responders at home or at a workplace. Furthermore, the cost of non-invasive procedures may be significantly reduced relative to comparable invasive procedures.

In some cases, the individual can apply the stimulator without the benefit of having a trained healthcare provider nearby. An advantage of this “self-stimulation” therapy is that it can be administered more or less immediately when symptoms occur, rather than having to visit the healthcare provider at a clinic or emergency room. A need for such a visit would only compound the aggravation that the individual is already experiencing. Another advantage of the self-stimulation therapy is the convenience of providing the therapy in the individual's home or workplace, which eliminates scheduling difficulties, for example, when the nerve stimulation is being administered for prophylactic reasons at odd hours of the day. Furthermore, the cost of the treatment may be reduced by not requiring the involvement of a trained healthcare provider.

Applicant has discovered that it is not necessary to “continuously stimulate” the vagus nerve in order to provide clinically efficacious benefits to individuals. The term “continuously stimulate” as defined herein means stimulation that follows a certain On/Off pattern continuously 24 hours/day. For example, existing implantable vagal nerve stimulators “continuously stimulate” the vagus nerve with a pattern of 30 seconds ON/5 minutes OFF (or the like) for 24 hours/day and seven days/week. Applicant has determined that this continuous stimulation is not necessary to provide the desired benefits described herein.

In certain embodiments, the methods and devices alter, modify or otherwise change the state of microglia in the central nervous system of the individual, such as in the brain. In particular, the devices and methods alter the microglia from a substantially pro-inflammatory state to a substantially non-inflammatory state.

The non- or anti-inflammatory state of microglia, also referred to as the quiescent or neuroprotective state, is generally constructive and supportive of neural growth and enhanced functional capacity of the network. Altering the state of microglia from the inflammatory state to the non-inflammatory state promotes neurogenesis, increases appropriate connectivity (which can be realized either by pruning inappropriate synapses or by enhancing synaptic formation and the maintenance and reinforcement of appropriate synapses) between neurons and improves the overall environment of the neural network. On the other hand, while the inflammatory state of microglia is generally to protect and defend the overall system from damage caused by external pathogens, it is accomplished at the expense of the resident tissue as it can be cytotoxic and destructive.

Applicant has discovered that the devices and methods described herein can reduce inflammation and drive microglia into the non-inflammatory, neurotrophic and/or quiescent state. That is, the microglia are induced by these methods into a state that is more ramified and into an activity that is more conducive to surveilling and synaptic pruning and reinforcing, which are necessary for learning and for creating the potential for learning and memory formation. This effect is, therefore, ideal for brain growth and optimal efficiency (e.g., memory creation, organization, and recall, mood and stress management, and alertness). Under normal physiological conditions, microglia mainly exist in a quiescent state and constantly monitor their microenvironment and survey neuronal and synaptic activity. Through the “Eat Me” (e.g., C1q, C3 and CR3) and “Don't Eat Me” (CD47 and SIRPα) pathways, as well as other pathways such as CX3CR1 signaling, non-inflammatory microglia regulate synaptic pruning, a process crucial for the promotion of synapse formation and the regulation of neuronal activity and synaptic plasticity. By mediating synaptic pruning, resting microglia play an important role in the regulation of learning and memory, including the modulation of memory strength, forgetfulness, and memory quality. See Microglia Regulation of Synaptic Plasticity and Learning and Memory, Cornell, Salinas, Huang and Zhou, Neural Regeneration Research, Vol. 17, No. 4, April 2022, the complete disclosure of which is incorporated herein by reference in its entirety for all purposes.

Recent studies have demonstrated many key roles for microglia in the formation and remodeling of neural circuits throughout several regions of the brain, particularly when the microglia are oriented towards their noninflammatory state. For example, studies have shown that microglia are critical to the survival, proliferation and maturation of neural progenitor cells (NPCs) in the developing brain. Microglia have also been shown to promote myelination by providing iron, a necessary co-factor for myelination to oligodendrocytes. There is also mounting evidence that microglia regulate the vascularization of the nervous system. Microglia have also been implicated in the remodeling of developing synapses in response to changes in neural activity. In addition to modulating development of existing connectivity, microglia have also been implicated in the initial wiring of the embryonic brain. See, for example, Microglia: Architects of the Developing Nervous System, Jeffrey L. Frost and Dorothy P. Schafer, Trends in Cell Biology, the complete disclosure of which is incorporated herein by reference in its entirety for all purposes.

Other studies have shown that microglia in the healthy brain habitually interact with neuronal and nonneuronal elements, both structurally and functionally. Forms of interactions include phagocytosis of synaptic structures during postnatal development, phagocytosis of newborn neurons during adult neurogenesis, and active remodeling of the peri synaptic environment and release of soluble factors in the mature and aging brain. These interactions can influence neuronal plasticity and function directly or indirectly, and may be regulated by sensory experience. Therefore, beyond their decisive role in pathological conditions, microglia are emerging as important contributors to normal brain physiology. See for example, The Role of Microglia in the Healthy Brain, Tremblay, Stevens, Sierra, Wake, Bessis and Nimmerjahn, The Journal of Neuroscience, Nov. 9, 2011: 34(45): 16064-16069, the complete disclosure of which is incorporated herein by reference in its entirety for all purposes.

Other studies have shown that microglia actively contribute to neuronal plasticity. Microglia regulate synaptic plasticity and the structural and functional changes in the brain that result from disrupted microglia-synapse interactions. Because microglia have dynamic responses and partake in synaptic plasticity during homeostasis, altered microglia physiology during stressful conditions could contribute to the pathophysiology of mental health and neurological disorders. See, for example, Microglia in Neuronal Plasticity: Influence of Stress, Delpech, Madore, Nadjar, Joffre, Wohleb and Laye, Neuropharmacology 96 (2015) 19-28 and Microglia Regulate Synaptic Development and Plasticity, Andoh and Koyama, Laboratory of Chemical Pharmacology, Graduate School of Pharmaceutical Sciences, the University of Tokyo, DOI: 10.1002/dneu.22814, the complete disclosures of which are incorporated herein by reference in their entirety for all purposes.

In each of these studies, and similar related research, it has been revealed that inflammation, whether necessary or pathologic, disrupt the neurodevelopmental functions of microglia, and are associated with life-long inhibited neural function, including but not limited to cognitive, emotional, social, and pain regulatory functions that are critical for quality and functional life.

In embodiments, the electrical impulse is sufficient to activate autonomic nervous system control over immune function, and decrease expression of proinflammatory cytokines, such as TNF-α, Interleukin (IL)-1, IL-6, IL-8, IL-11 and other chemokines. In particular, the electrical impulse is sufficient to reduce the expression of TNF-α and IL-1β which limits the disruption of the synthesis of multiple inhibitory neurotransmitters, alters the expression levels of key transporter proteins associated with multiple neurotransmitters, triggers the production of peptides that are associated with pain and vasoconstriction, and upregulates the expression of receptors for neural excitation. While TNF-α and IL-1β play a physiological role in controlling synaptic transmission and plasticity in the healthy CNS by modulating ionotropic glutamate receptors tracking, excessive pro-inflammatory cytokine levels, as a result of different types of injury, have an inhibitory effect on glutamate transporters, resulting in increased glutamate concentration in the CNS. See Tumor Necrosis Factor Alpha: A Link Between Neuroinflammation and Excitotoxicity, Olmos and Llado, Hindawi Publishing Corporation, Mediators of Inflammation, Volume 2014, Article ID 861231, Cytokines as Biomarkers in Depressive Disorder: Current Standing and Prospects, Lichtblau, Schmidt, Schumann, Kirkby and Himmerich, International Review of Psychiatry, October 2013; 25(5): 592-603, and The Pro-Inflammatory Cytokine TNF-α Regulates the Activity and Expression of the Serotonin Transporter (SERT), Malynn, Campos-Torres, Moynagh and Haase, Neurochem Res. (2103) 38:694-704, the complete disclosures of which are incorporated herein by reference in their entirety for all purposes.

In embodiments, the electrical impulse is sufficient to increase the expression of anti-inflammatory cytokines, such as IL-1ra or a CX3CL1 chemokine, IL-1 receptor antagonist, IL-4 IL-10, IL-11, and IL-13 and the like. In particular, the electrical impulse increases the expression of IL-1ram, CX3CL1 chemokines, brain-derived neurotrophic factors (BDNF).

The canonical cholinergic anti-inflammatory pathway, or CAP, involves the release of acetylcholine and the corresponding activation of the α7 nicotinic acetylcholine receptor, or α7nAChR, which triggers an intracellular pathway that suppresses the expression of the entire inflammasome (the set of pro-inflammatory genes, including TNF-α). This vagally-mediated pathway is either active and anti-inflammatory, or is not active and has little to no effect on the immune cells.

Alternatively, vagal nerve stimulation as described herein may be a secondary means of control compared with a noradrenergic pathway mediated β1 and βλ receptors expressed on macrophages and monocytes. According to this embodiment, norepinephrine released by sympathetic nerves have the ability to both upregulate and downregulate the activity of these immune cells, and thus serve as bidirectional controllers of the immune system.

The effects of VNS according to the system and methods described herein to the central nervous system is similar regardless of which of these two mechanisms are in effect, or if both are in effect. In the afferent direction, the vagus nerve enters the brain through the nucleus tractus solitarius, which is a bilateral set of elongate structures deep within the brain stem, having connections to many of the most fundamental neurotransmitter structures, including (i) the locus coeruleus (LC), which is the sole source of norepinephrine in the CNS, and (ii) the nucleus basalis of Meynert (NBM), which is a primary source of acetylcholine in the CNS. Electrical stimulation of the vagus nerve causes the release of acetylcholine from the NBM and norepinephrine from the LC.

Treatment Paradigms

The present description contemplates at least two types of interventions involving stimulation of a vagus nerve: acute and chronic. The acute treatment involves the fewest administrations of vagus nerve stimulations and is intended primarily to enlist and engage the autonomic nervous system to inhibit excitatory neurotransmissions. This treatment may be used, for example, over a shorter period, e.g., one day, two days, one week, one month, or three months, to promote enhanced speed of learning, superior recall, efficient application of learned information, prolonged alertness or less fatigue, better mood and/or more restful and restorative sleep. The acute stimulation protocol provides temporary improvement to enable an individual to, for example, accomplish a particular task while under duress, sleep deprivation or the like.

A vagus nerve stimulation treatment is conducted for continuous period of thirty seconds to five minutes, preferably about 90 seconds to about three minutes and more preferably about two minutes (each defined as a single dose). After a dose has been completed, the therapy is stopped for a period of time (depending on the treatment as described below). In exemplary embodiments, the acute treatment comprises one of the following: (1) 2-12 doses/day, preferably about 2-4 doses, at predetermined intervals or times; (2) two doses, either consecutively, or separated by 5 min at predetermined intervals or times, preferably two to four times/day; (3) 3 doses, either consecutively or separated by 5 min again at predetermined intervals or times, such as 2 or 3 times/day; or (4) 1-3 doses, either consecutively or separated by 5 min, 4-6 times per day.

In particular, Applicant has discovered that the stimulation protocol described herein may increase cognitive performance under sleep deprivation stress. Fatigue is a serious and unavoidable problem for many professions such as medicine, transportation, and the military. Fatigue induced by sustained wakefulness can cause slower reaction times, reduced ability to multi-task, and increases in lapses of attention which can lead to costly, even deadly mistakes. Studies on the behavioral implications of repeated sleep deprivation in humans have showed delayed reaction times, decreased accuracy and attention, and negative alterations in mood. Although some pharmacological fatigue countermeasures do exist, they vary in their effectiveness, have a range of negative side effects, and may lose effectiveness with repeated use.

Applicant has performed a randomized double-blind, sham-controlled trial at Wright Patterson Air Force Base using the devices described herein. The study, sponsored by the US Air Force Research Laboratory, treated 40 United States Air Force personnel and investigated the use of nVNS as a fatigue countermeasure. This study was the first to assess cognitive performance during nVNS, and the first to do so under conditions of lengthy sleep deprivation. See Cervical Transcutaneous Vagal Nerve Stimulation (ctVNS) Improves Human Cognitive Performance Under Sleep Deprivation Stress, L. McIntire, McKinley, Goodyear, J. McIntire and Brown, Communications Biology (2021) 4:634, the complete disclosure of which is incorporated herein by reference in its entirety for all purposes.

Study participants were awake for 34 consecutive hours and tested at multiple time points assessing the effects of sleep deprivation on their ability to multi-task. The group treated with nVNS showed a statistically significant benefit in their throughput capacity when compared to the sham group, with the nVNS treated group's throughput capacity decreasing by 5% from baseline vs. the sham group, which fell 15% (p<0.001). Furthermore, six stimulations of nVNS (12 minutes total) provided benefit to multi-tasking performance up to 15 hours post-stimulation, when performance should be at its worst. The study also found the nVNS group had a significantly smaller increase in a subjective fatigue rating when compared to sham participants (p<0.001).

Applicant has also conducted a study related to enhancing training of military personnel. This study demonstrated that the stimulation protocols described herein produced 20% acceleration in training along with a 35% improvement in memory retention during intelligence, surveillance and reconnaissance (ISR) training. Participants in the study also exhibited a 25% improvement in attention and mood.

For chronic treatment, such as treatments to permanently enhance the intelligence, emotional stability, and overall brain health of the treated individual, the therapy preferably comprises multiple doses/day over a period of time that may last from one day to a number of months or even years. Applicant has discovered that applying the stimulation protocols described herein over a period of years provides a permanent increase in the overall brain health of the individual, which can lead to increase in intelligence, learning capacity, memory retention and/or emotional stability of the individual.

In particular, Applicant has discovered that these stimulation protocols are particularly useful when applied to children that are still undergoing brain development. Studies have shown that microglia are the key players for synaptic regulation required for brain function in developing and adult brains. For example, microglia can sense synaptic activity via several putative mechanisms and modify morphology and function of synaptic terminals to regulate the neural circuit to maintain optimal conditions. See, for example, Microglia: Lifelong Modulator of Neural Circuits, Ikegami, Haruwaka and Wake, Neuropathology 2019: 39, 173-180, the complete disclosure of which is incorporated herein by reference in its entirety for all purposes.

For example, in some embodiments, the stimulation protocols are applied to children under the age of 18, or under the age of 10, or under the age of 5 or even under the age of 2 years old. The stimulation protocols may be applied for a period of at least six months, or one year, preferably for five or more years during the period of childhood development. This results in a permanent change in the neurostructural development of the child.

For certain individuals, the time of day can be more important than the time interval between treatments. For example, the locus correleus has periods of time during a 24 hour day wherein it has inactive periods and active periods. Typically, the inactive periods can occur in the late afternoon or in the middle of the night when the individual is asleep. It is during the inactive periods that the levels of inhibitory neurotransmitters in the brain that are generated by the locus correleus are reduced. This may have an impact on certain treatments.

In these embodiments, the chronic treatment may comprise multiple doses/day timed for periods of inactivity of the locus correleus. In one embodiment, a treatment comprises one or more doses administered 2-4 times per day or 2-4 “treatment sessions” per day, preferably about 2 treatment sessions per day. The treatment sessions preferably occur during the late afternoon or late evening, in the middle of the night and again in the morning when the individual wakes up. In an exemplary embodiment, each treatment session comprises 1-4 doses, preferably 1-2 doses, with each dose lasting for about 60 seconds to about 5 minutes, preferably about 90 seconds to about three minutes.

For other individuals, the intervals between treatment sessions may be the most important as applicant has determined that stimulation of the vagus nerve can have a prolonged effect on the inhibitor neurotransmitters levels in the brain, e.g., at least one hour, up to 3 hours and sometimes up to 8 hours. In one embodiment, a treatment comprises one or more doses (i.e., treatment sessions) administered at intervals during a 24 hour period. In a preferred embodiment, there are 1-5 such treatment sessions, preferably 2-4 treatment sessions. Each treatment session preferably comprises 1-3 doses, each “dose” lasting between about 60 seconds to about five minutes, preferably about 90 seconds to about 150 seconds, more preferably about 2 minutes.

For all of the treatments listed above, one may alternate treatment between left and right sides, or in the case of stroke or migraine that occur in particular brain hemispheres, one may treat ipsilateral or contralateral to the stroke-hemisphere or headache side, respectively. Or for a single treatment, one may treat one minute on one side followed by one minute on the opposite side. Variations of these treatment paradigms may be chosen on an individual-by-individual basis. However, it is understood that parameters of the stimulation protocol may be varied in response to heterogeneity in the symptoms of individuals. Different stimulation parameters may also be selected as the course of the individual's condition changes.

Description of Various Nerve Stimulating/Modulating Devices

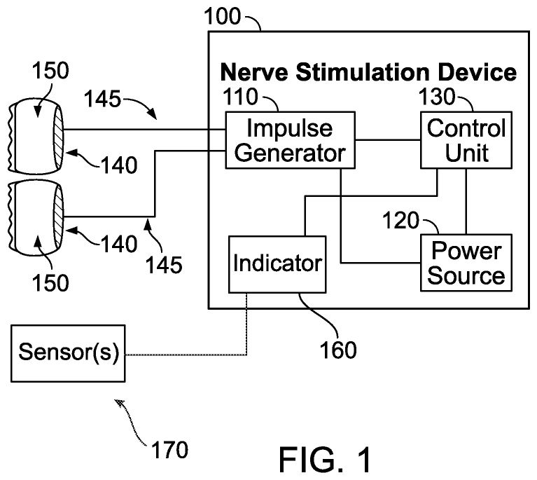

Referring now to FIG. 1, an electrode-based nerve stimulating and/or modulating device 100 is provided for delivering impulses of energy to nerves for the treatment of medical conditions. As shown, device 100 may include an impulse or signal generator 110, an energy or power source 120 coupled to the impulse generator 110 and/or a control unit 130 in communication with the impulse generator 110 and coupled to the energy source 120. Device 100 further includes one or more electrodes 140 coupled via wires 145 (or wirelessly) to impulse generator 110. Alternatively, electrodes 140 may be housed within, or on the outer surface of, device 100.

In some embodiments, the same impulse generator 110, energy source 120, and control unit 130 may be used for either a magnetic stimulator or an electrode-based stimulator, allowing the user to change parameter settings depending on whether magnetic coils or the electrodes 140 are attached.

Although a pair of electrodes 140 is shown in FIG. 1, in practice the electrodes may also comprise one electrode, or three or more distinct electrode elements, each of which is connected in series or in parallel to the impulse generator 110. Thus, the electrodes 140 that are shown in FIG. 1 represent some, most, many, or all electrodes of the device collectively.

Electrodes 140 may include a suitable adhesive that secured them to a skin surface. Suitable adhesive electrodes for use herein may include electrode pads, self-adhesive electrodes or the like. In this embodiment, electrodes 140 may be placed in a suitable location on the individual's neck and adhered thereto. Electrodes 140 receive electrical impulses from pulse generator 110. The duration, amplitude, frequency and treatment paradigm for the electrical impulses may be controlled by controller 130, a mobile device, or via another electronic device coupled to pulse generator 110. This embodiment allows, for example, a physician to secure electrodes 140 to the individual's neck such that the treatment paradigm may be followed without individual involvement. This is particularly useful for treating individuals that are unable or unwilling to self-treat. For example, in some cases, individuals recovering from surgery, such as major colorectal surgery may be either incapable of self-treatment, or their compliance with the treatment protocol may not be complete. In another example, older individuals may not have suitable mental faculties for self-treatment.

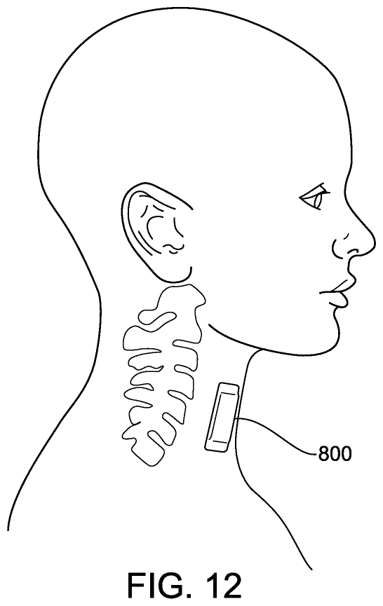

Stimulator 100 may be housed in an outer covering or patch 800 to protect stimulator from the environment (see FIG. 12). The patch may include a suitable adhesive strip or pad on one surface for adhering the patch and stimulator to the outer skin surface of the individual.

The stimulator in this embodiment includes one or more electrodes. The stimulator may also include a power source such as a battery, and a signal generator for applying the electrical impulses to the electrodes. Alternatively, the power source and/or the signal generator may be wirelessly coupled to the electrodes, as discussed above. An external controller may be wirelessly coupled to the stimulator to provide a stimulation protocol to the signal generator and to control other key functions of the signal, such as power, amplitude, duration frequency and the like.

The stimulator may reside in a housing that is removably coupled to the patch via a snap-fitting, Velcro, or other suitable attachment means. In this embodiment, the patch may be adhered to the individual and the stimulator may be removed and reattached without removing the patch. This allows the healthcare professions to, for example, recharge the battery, troubleshoot the device and/or control the stimulation therapy on the device.

The stimulator may also include a conductive fluid, such as a gel pad, disposed between the electrode(s) and the individual's outer skin surface to enhance conductivity of the electrical impulses through the outer skin surface to the nerve.

Alternatively, the outer covering may comprise any wearable material that may include the stimulator. For example, depending on the location of the target nerve on the individual's body, the stimulator may be attached to, or embedded within, a wearable garment, such as a shirt, scarf, watch, hat, gloves, pants, shoes, boots, socks, underwear, belt, dress, jacket, sweater, ear muffs, or the like. The wearable garment may also comprise an accessory, such as a wristband, ankle or wrist bracelet, necklace, earrings, a compression garment, an ankle or knee brace or the like.

In yet another embodiment, the garment itself is the stimulator. For example, the garment may comprise an electronic textile or e-textile that includes fabrics that enable digital components, such as electrodes, pulse generators, batteries wireless receivers and other electronic components to be embedded therein. Electronic textiles are distinct from wearable garments because the emphasis is placed on the seamless integration of textiles with electronic elements like microcontrollers, sensors, and actuators. In one embodiment, the electronic textile may comprise an organic electronics material that is conducting and has insulated electrical components that allows the garment to be washed without damaging the electronic components.

The stimulator may also include an array of electrodes. The electrode array may include multiple sets of electrodes with each set of electrodes configured to apply electrical impulses through the outer skin surface of the individual, as discussed above. Each of the sets of electrodes may be individually coupled to the pulse generator, either directly, through wires, or wireless as described above. The electrode array may have multiple patterns. For example, the array may be linear, square, circular or any other suitable shape.

In certain embodiments, the electrode array comprises two or more sets of electrodes, each spaced apart from each other between about 2 mm to about 25 mm, preferably between about 4 mm to about 10 mm. The electrode array preferably comprises a shape that substantially corresponds to a target area of the individual's neck. In one embodiment, the target area is the area on the neck that allows for electrical impulses to be passed through the skin to the vagus nerve (discussed in detail below).

The electrode sets may each be individually coupled to the pulse generator and/or the controller such that electrical impulses can be applied to all of the electrode sets, some of the electrode sets or only one of the electrode sets. In certain embodiments, the controller is configured to apply electrode impulses to only those electrodes positioned optimally for stimulating the nerve. In addition, the selection of electrodes may be dynamic and change over time.

In one such embodiment, the electrodes are arranged in an array or matrix that may contain tens to hundreds of microelectrodes. The microelectrodes may each be independently coupled to the pulse generator 110 such that the pulse generator can apply current to any one or a plurality of the microelectrodes. In some embodiments, groups of the microelectrodes are coupled together and then coupled to the pulse generator 110 such that electric current can be applied independently to each group. In an exemplary embodiment, the electrodes have a size of about 0.5 to 2.0 mm, preferably about 1.0 mm, and are spaced from each other a distance of about 0.5 to about 10 mm, preferably between about 2.0 mm and 5.0 mm (e.g., 3.0 mm).

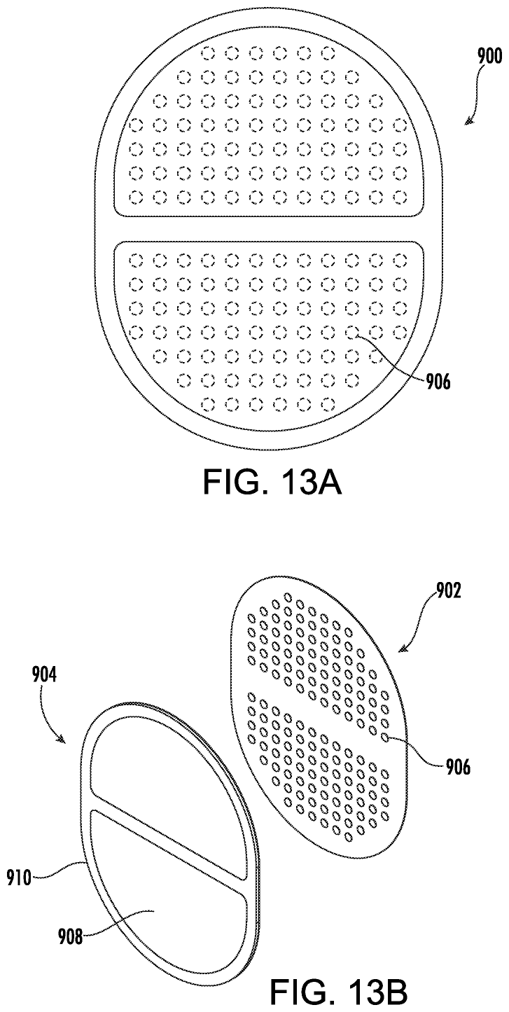

FIGS. 13A and 13B illustrate one example of an electrode array 900 that includes a flexible PCB board 902 and a cover 904. The PCB board 902 may comprise any number of microelectrodes 906 that are coupled to pulse generator 110 as described above. In the representative embodiment, the PCB board 902 includes about 20 to about 1,000 microelectrodes 906, or between about 40 to about 200 microelectrodes 906. In certain embodiments, cover 904 may include a conductive surface 908 overlying electrodes 906 and a non-conductive surface 910 overlying the remaining portions of the PCB board 902. Conductive surface 908 may include a conductive gel (not shown) and non-conductive surface may include a non-conductive gel.

Electrode array 900 may be included on the housing of a stimulator device, such as those described below. Alternatively, array 900 may be included as part of a patch, such as the patch 800 shown in FIG. 12 and discussed below. The dimensions of PCB board 902 will largely depend on the dimensions of the target region of the skin surface on the individual. In certain embodiments, the dimensions will be selected to encompass a region of the outer skin surface of the neck that is near the carotid sheath of the individual.

Stimulator 100 further comprises one or more sensors 170 coupled to stimulator 100 and/or electrodes 140 (or the microelectrodes in the array) for detecting whether the nerve has been stimulated, the amplitude of the stimulation, or whether the nerve has been stimulated with sufficient amplitude and other parameters to fire an action potential. The sensors 170 may detect a physiological parameter of the individual. Physiological parameters may include, for example, blood flow associated with a nerve, such as vagal artery or cerebral blood flow, heart rate or variability, ECG, respiration depth and rate, core temperature, hydration, blood pressure, brain function, oxygenation, skin impedance, and skin temperature, pupil diameter (e.g., pupil dilation), galvanic skin response, selected biomarkers or other chemicals, a property of a voice of the individual, a laryngeal electromyographic signal, an electroglottographic signal, a property of the autonomic nervous system and the like. Alternatively, the sensors 170 may be coupled to the electrodes 140 and may sense one or more parameters of the electrodes, such as impedance, amplitude, voltage or the like.

The sensors 170 may also be coupled to the controller 130. In this embodiment, the controller 130 is configured to receive input from the sensors and to direct the pulse generator 110 to apply electrical impulses to one or more sets of the electrodes 140 based on this input. For example, the sensors 170 may provide data that suggests that one or more of the sets of electrodes is not positioned properly to stimulate the nerve, or to stimulate the nerve at the optimal signal strength to cause the nerve to fire an action potential. The controller 130 is configured to shift the electrical impulse to the set or sets of electrodes that provide a sufficient electrical impulse to the nerve to cause it to fire an action potential. In this manner, the controller 130 can optimize the application of the electrical impulses to the nerve.

Sensor(s) 170 may be coupled to electrodes 140, or they may be formed as part of the electrodes 140. Alternatively, sensor(s) 140 may be only coupled to stimulator 100, or they may be coupled to a separate device, such as a mobile device (discussed below). In certain embodiments, stimulator 100 will comprise a housing that includes both electrodes 140 and sensors 170, as discussed in more detail below.

In certain embodiments, sensor(s) 170 are configured to detect a target position for stimulating a selected nerve within an individual. The target position may, for example, be located on an outer skin surface of the individual and the selected nerve may be located within the individual under the skin surface. In some cases, the selected nerve may be located deep within the individual, i.e., greater than 5 mm below the outer skin surface, greater than 10 mm, or even greater than 20 mm below. In one such embodiment, the selected nerve is the vagus nerve and the target location is a position on the outer skin surface of the neck of the individual suitable for passing an electrical impulse through the skin sufficient to modulate the vagus nerve.

In one embodiment, sensor 170 comprises a heart pulse sensor configured to detect a heart pulse in the individual. The heart pulse sensor may be any suitable sensor known in the art for detecting the heart pulse of an individual, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 170 is located adjacent to, or near, the individual's heart pulse.

The heart pulse sensor is preferably designed to contact the individual's outer skin surface and detect a pulse adjacent to, or near the sensor. However, in certain embodiments, the heart pulse sensor may be designed to detect the heart pulse without contacting the skin surface, e.g., through vibration, sound or other detection mechanisms. In these embodiments, sensor 170 may, for example, be located within stimulator 100, or within a separate device.

Sensors 170 may be coupled to an indicator 160 within stimulator 100, or within a separate device, such as a mobile device (discussed in more detail below). Indicator 160 is configured to generate an alert when sensors 170 have detected the target nerve. The alert may be, for example, a visual, tactile or audial alert, that provides the user with an indication that the sensor 170 has detected the target location.

In one such embodiment, sensor 170 comprises a heart pulse sensor that is configured to detect a heart pulse emanating from the carotid artery in the individual's neck. The vagus nerve is situated within the carotid sheath, near the carotid artery and the interior jugular vein. The carotid sheath is located at the lateral boundary of the retropharyngeal space on each side of the neck and deep to the sternocleidomastoid muscle.

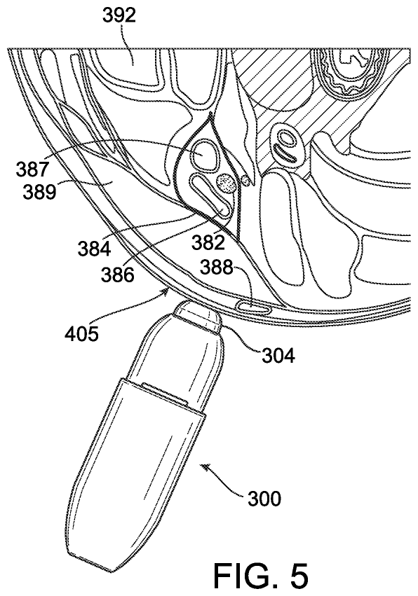

As discussed in detail below in reference to FIG. 5, the three major structures within the carotid sheath 384 are the common carotid artery 387, the internal jugular vein 388 and the vagus nerve 382. The carotid artery lies medial to the internal jugular vein, and the vagus nerve is situated posteriorly between the two vessels. Proceeding from the skin of the neck above the sternocleidomastoid muscle to the vagus nerve, a line may pass successively through the sternocleidomastoid muscle, the carotid sheath and the internal jugular vein, unless the position on the skin is immediately to either side of the external jugular vein. In the latter case, the line may pass successively through only the sternocleidomastoid muscle and the carotid sheath before encountering the vagus nerve, missing the interior jugular vein. Accordingly, a point on the neck adjacent to the external jugular vein might be preferred for non-invasive stimulation of the vagus nerve.

Sensors 170 are configured to detect the heart pulse emanating from the carotid artery to provide an indication that electrodes 150 are located adjacent to, or near the carotid sheath and/or the external jugular vein and thus near the vagus nerve. This provides confirmation to the user that the device is positioned optimally for stimulating the vagus nerve.

In certain embodiments, sensors 170 may be configured to detect a magnitude of the heart pulse emanating from the carotid artery. In these embodiments, the sensors 170 may be configured, for example, to only provide an indication that the heart pulse has been detected when the magnitude of heart pulse reaches a threshold level, indicating that the sensor is close to the carotid artery. Alternatively, the sensors 170 may transmit the magnitude of heart pulse detected to a controller or suitable electronics within stimulator, or a separate mobile device.

In certain embodiments, indicator 160 is configured to transmit an alert that is associated with the magnitude of the heart pulse. For example, the alert may comprise an audible sound that increases in decibel level as the magnitude increase. In another example, the alert may comprise a vibration that increases in intensity or frequency as the magnitude of the heart pulse increases. In yet another example, the alert may comprise a visual signal, such as a blinking light that increases in intensity with heart pulse magnitude, different colored lights associated with threshold magnitudes of heart pulse, or another visual signal, such as bars, lines or other shapes that increase in size (e.g., length or width) with increasing heart pulse magnitude.

The indicator 160 may further be configured to provide a second alert when the magnitude of the heart pulse reaches a threshold level associated with optimal positioning of the sensor 170 and/or the electrodes 150. For example, if the indicator is providing a blinking light that increases in intensity with heart pulse magnitude, the second alert may be that the blinking light stops blinking and becomes constant, or it changes color (e.g., from yellow to green), or a separate alert is produced, such as a sound, vibration or the like.

In this embodiment, the sensor 160 may comprise a heart pulse sensor configured to contact the outer skin surface of the individual and directly detect the pulse within the carotid sheath, such as an infrared sensor, optical sensor, tactile sensor, a photoplethysmography (PPG) sensor or the like.

Alternatively, the sensor 160 may comprise an ultrasound transducer or probe configured to detect the location of the vagus nerve underlying 100 stimulator. The probe may be housed within stimulator 100, or it may be a separate device. The probe may be connected to an ultrasound machine that displays the anatomical structures that lie under the probe. Alternatively, the probe may be coupled to a controller or other device that is configured to provide an indication or alert when the probe has illustrated the carotid sheath.

The control unit 130 controls the impulse generator 110 to generate a signal for each of the device's electrodes (or magnetic coils). The signals are selected to be suitable for amelioration of a particular medical condition when the signals are applied non-invasively to a target nerve or tissue via the electrodes 140. It is noted that nerve stimulating/modulating device 100 may be referred to by its function as a pulse generator. Patent application publications US2005/0075701 and US2005/0075702, both to SHAFER, the disclosure of which is incorporated herein by reference for all purposes as if copied and pasted herein, contain descriptions of pulse generators that may be applicable to this disclosure. By way of example, a pulse generator is also commercially available, such as Agilent 33522A Function/Arbitrary Waveform Generator, Agilent Technologies, Inc., 5301 Stevens Creek Blvd Santa Clara CA 95051.

The control unit 130 may comprise a general purpose computer, comprising one or more CPU, computer memories for the storage of executable computer programs (including the system's operating system) and the storage and retrieval of data, disk storage devices, communication devices (such as serial and USB ports) for accepting external signals from a keyboard, computer mouse, and touchscreen, as well as any externally supplied physiological signals, analog-to-digital converters for digitizing externally supplied analog signals, communication devices for the transmission and receipt of data to and from external devices such as printers and modems that comprise part of the system, hardware for generating the display of information on monitors or display screens that comprise part of the system, and busses to interconnect the above-mentioned components. Thus, the user may operate the system by typing or otherwise providing instructions for the control unit 130 at a device such as a keyboard or touch-screen and view the results on a device such as the system's computer monitor or display screen, or direct the results to a printer, modem, and/or storage disk. Control of the system may be based upon feedback measured from externally supplied physiological or environmental signals. Alternatively, the control unit 130 may have a compact and simple structure, for example, wherein the user may operate the system using only an on/off switch and energy control wheel or knob, or their touchscreen equivalent. In a section below, an embodiment is also described wherein the stimulator housing has a simple structure, but other components of the control unit 130 are distributed into other devices.

Parameters for the nerve or tissue stimulation include energy level, frequency and train duration (or pulse number). The stimulation characteristics of each pulse, such as depth of penetration, strength and selectivity, depend on the rise time and peak electrical energy transferred to the electrodes, as well as the spatial distribution of the electric field that is produced by the electrodes. The rise time and peak energy are governed by the electrical characteristics of the stimulator and electrodes, as well as by the anatomy of the region of current flow within the individual. In some embodiments, pulse parameters are set in such a way as to account for the detailed anatomy surrounding the nerve that is being stimulated [Bartosz SAWICKI, Robert Szmurio, Przemyslaw Plonecki, Jacek Starzynski, Stanislaw Wincenciak, Andrzej Rysz. Mathematical Modelling of Vagus Nerve Stimulation. pp. 92-97 in: Krawczyk, A. Electromagnetic Field, Health and Environment: Proceedings of EHE '07. Amsterdam, 105 Press, 2008, the disclosure of which is incorporated herein by reference for all purposes as if copied and pasted herein]. Pulses may be monophasic, biphasic or polyphasic. In some embodiments, some devices include those that are fixed frequency, where each pulse in a train has the same inter-stimulus interval, and those that have modulated frequency, where the intervals between each pulse in a train can be varied.

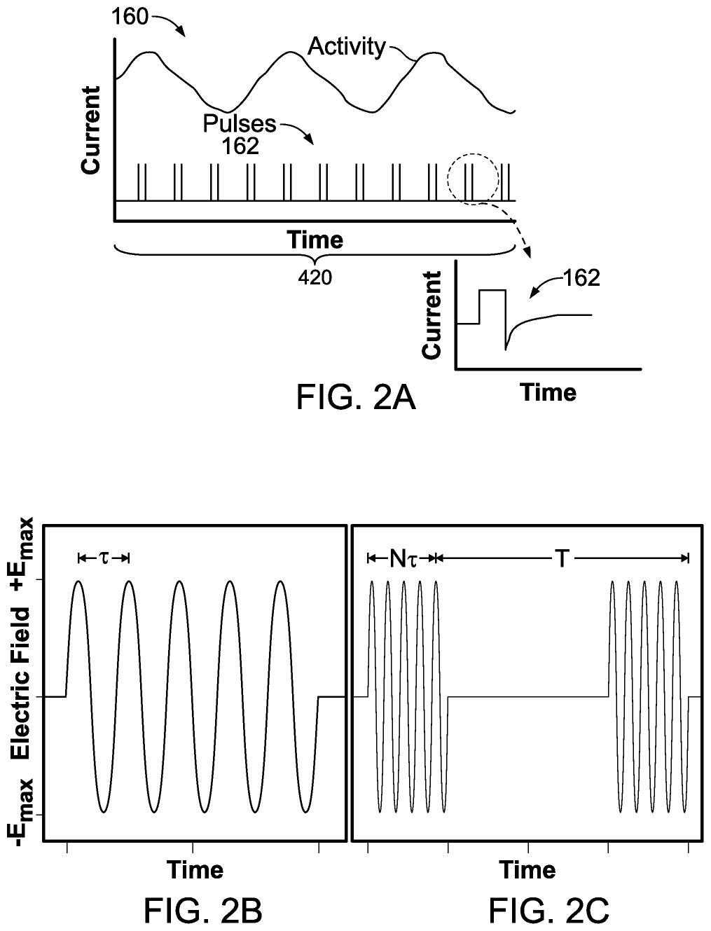

FIG. 2A illustrates an example of an electrical voltage/current profile for a stimulating, blocking and/or modulating impulse applied to a portion or portions of selected nerves in accordance with an embodiment of this disclosure. For some embodiments, the voltage and current refer to those that are non-invasively produced within the individual by the electrodes (or magnetic coils). As shown, a suitable electrical voltage/current profile 160 for the blocking and/or modulating impulse 162 to the portion or portions of a nerve may be achieved using pulse generator 110. In some embodiments, the pulse generator 100 may be implemented using an energy source 120 and a control unit 130 having, for instance, a processor, a clock, a memory, etc., to produce a pulse train 164 to the electrodes 140 that deliver the stimulating, blocking and/or modulating impulse 162 to the nerve. Nerve stimulating/modulating device 100 may be externally energized and/or recharged or may have its own energy source 120. The parameters of the modulation signal 160, such as the frequency, amplitude, duty cycle, pulse width, pulse shape, etc., can be programmable, non-programmable, modifiable, locally or remotely updateable, or others. An external communication device may modify the pulse generator programming to improve treatment.

In addition, or as an alternative to some of the devices to implement the modulation unit for producing the electrical voltage/current profile of the stimulating, blocking and/or modulating impulse to the electrodes, the device disclosed in US Patent Application Publication No. US2005/0216062, the disclosure of which is incorporated herein by reference for all purposes as if copied and pasted herein, may be employed. That patent publication discloses a multifunctional electrical stimulation (ES) system adapted to yield output signals for effecting electromagnetic or other forms of electrical stimulation for a broad spectrum of different biological and biomedical applications, which produce an electric field pulse in order to non-invasively stimulate nerves. The system includes an ES signal stage having a selector coupled to a plurality of different signal generators, each producing a signal having a distinct shape, such as a sine wave, a square or a saw-tooth wave, or simple or complex pulse, the parameters of which are adjustable in regard to amplitude, duration, repetition rate and other variables. Examples of the signals that may be generated by such a system are described in a publication by LIBOFF [A. R. LIBOFF. Signal shapes in electromagnetic therapies: a primer. pp. 17-37 in: Bioelectromagnetic Medicine (Paul J. Rosch and Marko S. Markov, eds.). New York: Marcel Dekker (2004), the disclosure of which is incorporated herein by reference for all purposes as if copied and pasted herein]. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated, as well as the outputs of various sensors which sense prevailing conditions prevailing in this substance, whereby the user of the system can manually adjust the signal, or have it automatically adjusted by feedback, to provide an electrical stimulation signal of whatever type the user wishes, who can then observe the effect of this signal on a substance being treated.

The stimulating and/or modulating impulse signal 160 preferably has a frequency, an amplitude, a duty cycle, a pulse width, a pulse shape, etc. selected to influence the therapeutic result, namely, stimulating and/or modulating some or all of the transmission of the selected nerve. For example, the frequency may be about 1 Hz or greater, such as between about 15 Hz to 100 Hz, preferably between about 15-50 Hz and more preferably between about 15-35 Hz. In some embodiments, the frequency is 25 Hz. The modulation signal may have a pulse width selected to influence the therapeutic result, such as about 1 microseconds to about 1000 microseconds, preferably about 100-400 microseconds and more preferably about 200-400 microseconds. For example, the electric field induced or produced by the device within tissue in the vicinity of a nerve may be about 5 to 600 V/m, preferably less than 100 V/m, and even more preferably less than 30 V/m. The gradient of the electric field may be greater than 2 V/m/mm. More generally, the stimulation device produces an electric field in the vicinity of the nerve that is sufficient to cause the nerve to depolarize and reach a threshold for action potential propagation, which is approximately 8 V/m at 1000 Hz. The modulation signal may have a peak voltage amplitude selected to influence the therapeutic result, such as about 0.2 volts or greater, such as about 0.2 volts to about 40 volts, preferably between about 1-20 volts and more preferably between about 2-12 volts.

In an exemplary embodiment, the waveform comprises bursts of sinusoidal pulses, as shown in FIGS. 2B and 2C. As seen there, individual sinusoidal pulses have a period of T, and a burst consists of N such pulses. This is followed by a period with no signal (the inter-burst period). The pattern of a burst followed by silent inter-burst period repeats itself with a period of T. For example, the sinusoidal period T may be between about 50-1000 microseconds with a frequency of about 1-20 kHz), preferably between about 100-400 microseconds with a frequency of about 2.5-10 kHz, more preferably about 133-400 microseconds with a frequency of about 2.5-7.5 kHz and even more preferably about 200 microseconds with a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2E to make the bursts discernable). When these exemplary values are used for T and τ, the waveform contains significant Fourier components at higher frequencies ( 1/200 microseconds=5000/sec), as compared with those contained in transcutaneous nerve stimulation waveforms, as currently practiced.

The above waveform is essentially a 1-20 kHz signal that includes bursts of pulses with each burst having a frequency of about 5-100 Hz and each pulse having a frequency of about 1-20 kHz. Another way of thinking about the waveform is that it is a 1-20 kHz waveform that repeats itself at a frequency of about 5-100 Hz.

Invasive nerve stimulation typically uses square wave pulse signals. However, Applicant found that square waveforms are not ideal for non-invasive stimulation, as they produce excessive pain, but still can be used. Prepulses and similar waveform modifications have been suggested as methods to improve selectivity of nerve stimulation waveforms, but Applicant also did not find them ideal, although they still can be used [Aleksandra VUCKOVIC, Marco Tosato and Johannes J Struijk. A comparative study of three techniques for diameter selective fiber activation in the vagal nerve: anodal block, depolarizing prepulses and slowly rising pulses. J. Neural Eng. 5 (2008): 275-286, the disclosure of which is incorporated herein by reference for all purposes as if copied and pasted herein; Aleksandra VUCKOVIC, Nico J. M. Rijkhoff, and Johannes J. Struijk. Different Pulse Shapes to Obtain Small Fiber Selective Activation by Anodal Blocking—A Simulation Study. IEEE Transactions on Biomedical Engineering 51(5,2004):698-706, the disclosure of which is incorporated herein by reference for all purposes as if copied and pasted herein; Kristian HENNINGS. Selective Electrical Stimulation of Peripheral Nerve Fibers: Accommodation Based Methods. Ph.D. Thesis, Center for Sensory-Motor Interaction, Aalborg University, Aalborg, Denmark, 2004, the disclosure of which is incorporated herein by reference for all purposes as if copied and pasted herein].

In some embodiments, the use of feedback to generate the modulation signal 160 may result in a signal that is not periodic, particularly if the feedback is produced from sensors that measure naturally occurring, time-varying aperiodic physiological signals from the individual. In fact, the absence of significant fluctuation in naturally occurring physiological signals from an individual is ordinarily considered to be an indication that the individual is in ill health. This is because a pathological control system that regulates the individual's physiological variables may have become trapped around only one of two or more possible steady states and is therefore unable to respond normally to external and internal stresses. Accordingly, even if feedback is not used to generate the modulation signal 160, it may be useful to artificially modulate the signal in an aperiodic fashion, in such a way as to simulate fluctuations that would occur naturally in a healthy individual. Thus, the noisy modulation of the stimulation signal may cause a pathological physiological control system to be reset or undergo a non-linear phase transition, through a mechanism known as stochastic resonance [B. SUKI, A. Alencar, M. K. Sujeer, K. R. Lutchen, J. J. Collins, J. S. Andrade, E. P. Ingenito, S. Zapperi, H. E. Stanley, Life-support system benefits from noise, Nature 393 (1998) 127-128, the disclosure of which is incorporated herein by reference for all purposes as if copied and pasted herein; W Alan C MUTCH, M Ruth Graham, Linda G Girling and John F Brewster. Fractal ventilation enhances respiratory sinus arrhythmia. Respiratory Research 2005, 6:41, pp. 1-9, the disclosure of which is incorporated herein by reference for all purposes as if copied and pasted herein].

In some embodiments, the modulation signal 160, with or without feedback, will stimulate the selected nerve fibers in such a way that one or more of the stimulation parameters (e.g., energy, frequency, and others mentioned herein) are varied by sampling a statistical distribution having a mean corresponding to a selected, or to a most recent running-averaged value of the parameter, and then setting the value of the parameter to the randomly sampled value. The sampled statistical distributions will comprise Gaussian and 1/f, obtained from recorded naturally occurring random time series or by calculated formula. Parameter values will be so changed periodically, or at time intervals that are themselves selected randomly by sampling another statistical distribution, having a selected mean and coefficient of variation, where the sampled distributions comprise Gaussian and exponential, obtained from recorded naturally occurring random time series or by calculated formula.

In some embodiments, some devices, as disclosed herein, are provided in a “pacemaker” type form, in which electrical impulses 162 are generated to a selected region of the nerve by a stimulator device on an intermittent basis, to create in the individual a lower reactivity of the nerve.

Embodiments of the Stimulators

The electrodes of the some of the devices, as disclosed herein, are applied to the surface of the neck, or to some other surface of the body, and are used to deliver electrical energy non-invasively to a nerve. Embodiments may differ with regard to the number of electrodes that are used, the distance between electrodes, and whether disk, ring or other shapes of electrodes are used. In some embodiments, one selects the electrode configuration for individual individuals, in such a way as to optimally focus electric fields and currents onto the selected nerve, without generating excessive currents on the surface of the skin.

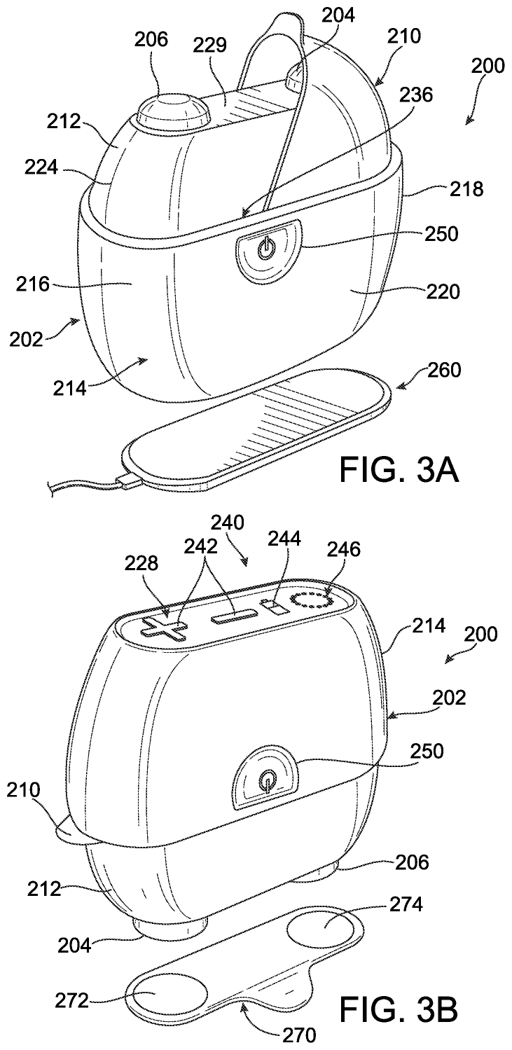

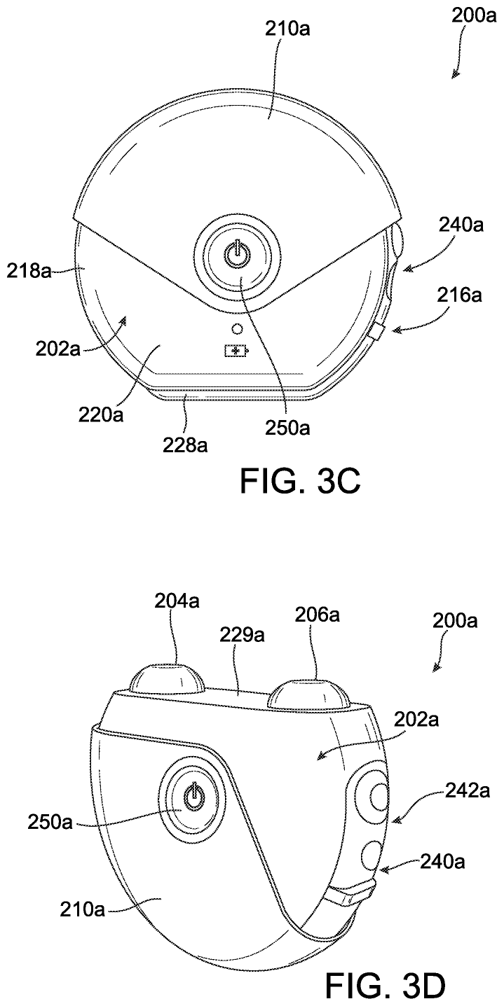

Referring now to FIGS. 3A and 3B, one embodiment of a stimulator 200 comprises a housing 202 and first and second electrodes 204, 206 extending from one surface of housing 202. Electrodes 204, 206 are applied to a surface of the individual's body, during which time stimulator 200 may be held in place by straps, frames, collars or the like, or the stimulator 200 may be held against the individual's body by hand.

Housing 202 contains the electronic components, signal generator and energy source (not shown) that are used to generate the signals that drive electrical impulses through electrodes 204, 206. However, in other embodiments, the electronic components that generate the signals may be in a separate housing or device, such as a mobile device. Furthermore, other embodiments may contain a single electrode or more than two electrodes.

Housing 202 comprises upper and lower portions 212, 214 and a cover 210 disposed between upper and lower portions 212, 214 for protecting electrodes 204, 206 from the external environment. Cover 210 also ensures that electrodes 204, 206 will not contact an individual's tissue when the device is not intended to be used (e.g., in the event that the device is accidently turned ON and electric current is passed through electrodes when not in use). In certain embodiments, cover 210 is rotatably coupled to housing 202 such that it can be moved between a first position, wherein the electrodes 204, 206 are exposed for stimulation, and a second position, wherein the electrodes are housed and protected within the cover 210. Cover 210 may comprise any suitable material, such as polyphenylene ether (PPE), plastic, or other polymers.

Lower portion 214 of housing 202 preferably includes curved side surfaces 216, 218 with substantially planar surfaces 220 therebetween to form an overall disc-like shape that is cut off on the upper and lower portions of the disc. Lower portion 214 also includes a substantially planar bottom surface 228 that includes a control panel 240 (discussed below).

Similarly, upper portion 212 of housing preferably comprises curved side surfaces 224 and a substantially planar upper surface 229. Electrodes 204, 206 extend outward from upper surface 229. Upper portion 212 has a smaller width and length as lower portion 214 to form a groove 236 therebetween. Cover 210 rotates within groove 236. Upper and lower portions 212, 214 are preferably coupled to each other within housing 202. Alternatively, they may be molded together and formed as an integral component.

Control panel 240 may include a number of user controls and/or device status indicators. In alternative embodiments, the controls and status indicators are located on a separate device, such as a mobile device, that is wirelessly (e.g. Bluetooth or the like) coupled to stimulator 200.

In a preferred embodiment, control panel 240 includes intensity controls 242 for controlling the level of intensity or amplitude of the electrical impulses generated by stimulation 200. Intensity controls 242 extend outward from lower surface 228 so that the user be tactically identify and control intensity controls 242.

Control panel 240 may further comprise a battery life indicator 244 and/or a dose duration indicator 246. These indicators may include, for example, LEDs or other light sources, to facilitate identification by the user. The dose duration indicator 246 provides an indication of the time remaining on a single dose of electrical stimulation. In certain embodiments, stimulation 200 is configured to automatically cease the generation of the electrical impulse when the duration of the single dose has been completed.

Stimulator 200 may further include a power control 250 for turning ON the device. Power control 250 may also include an LED or other light source for illuminating power control 250 when the device has been turned ON. In one embodiment, power control 250 is located on side surface 200, although it will be understood that power control 250 may be located on control panel 240 or elsewhere on stimulator 200.

In certain embodiments, stimulator 200 further includes a charging pad 260 coupled to a suitable connector for providing power to stimulator 200 and/or recharging the battery within stimulator 200. Charging pad 260 may comprise any suitable charging source, such as an inductive charging source that provides power via inductive transmission through the lower surface 228 of housing 202.

Stimulator 200 may further include a gel pad 270 that includes a conductive gel 272, 274 positioned to contact electrodes 204, 206 when gel pad 270 is positioned over upper surface 229. Gel pad 270 is configured to apply a coating of electrically conductive gel to the surfaces of electrodes 204, 206 to facilitate conduction of the electrical impulses through an outer skin surface of the individual.

The housing 202 may comprise plastic, metal, rubber, or other materials. The housing 202 may be rigid, elastic, resilient, or flexible. The housing 202 may be included in, or embodied as, a phone, a tablet, a laptop, a phone/tablet/laptop case, a patch, an adhesive bandage, a strip, an anklet, a belt, a bracelet, a necklace, a garment, a pad, a ring, a mattress, a pillow, a blanket, a robot, a surgical instrument, a stimulator, an infusion device, or others. The housing 202 may be embodied as described in US Patent Application Publication 20140330336 and U.S. Pat. Nos. 8,874,205, 9,174,066, 9,205,258, 9,375,571, and 9,427,581, all of which are incorporated entirely herein by reference for all purposes as if copied and pasted herein.

Electrodes 204, 206 may comprise a substantially solid conducting material (e.g., metal such as stainless steel, platinum, or a platinum-iridium alloy), which is possibly flexible in some embodiments. However, in other embodiments, the electrodes may have many other sizes and shapes, and they may be made of other materials. The electrodes preferably have a dome-shape with a rounded distal surface, although they may have the shape of a screw that is flattened on its tip. Pointing of the tip would make the electrode more of a point source, such that the equations for the electrical potential may have a solution corresponding more closely to a far-field approximation. Rounding of the electrode surface or making the surface with another shape will likewise affect the boundary conditions that determine the electric field.

In other embodiments, electrodes 204, 206 may be housed within housing 200. In these embodiments, housing includes an outer contact surface, such as a fluid permeable material that allows for passage of current through the permeable portions of the material. In these embodiments, a conductive medium (such as a gel) is preferably situated between the electrode(s) and the permeable interface. The conductive medium provides a conductive pathway for electrons to pass through the permeable interface to the outer surface of the interface and to the individual's skin.

In other embodiments, the interface is made from a very thin material with a high dielectric constant, such as material used to make capacitors. For example, it may be Mylar having a submicron thickness (preferably in the range about 0.5 to about 1.5 microns) having a dielectric constant of about 3. Because one side of Mylar is slick, and the other side is microscopically rough, two different configurations are contemplated: one in which the slick side is oriented towards the individual's skin, and the other in which the rough side is so-oriented. Thus, at stimulation Fourier frequencies of several kilohertz or greater, the dielectric interface will capacitively couple the signal through itself, because it will have an impedance comparable to that of the skin. Thus, the dielectric interface will isolate the stimulator's electrode from the tissue, yet allow current to pass. In one embodiment, non-invasive electrical stimulation of a nerve is accomplished essentially substantially capacitively, which reduces the amount of ohmic stimulation, thereby reducing the sensation the individual feels on the tissue surface. This would correspond to a situation, for example, in which at least 30%, preferably at least 50%, of the energy stimulating the nerve comes from capacitive coupling through the stimulator interface, rather than from ohmic coupling. In other words, a substantial portion (e.g., 50%) of the voltage drop is across the dielectric interface, while the remaining portion is through the tissue.

In certain embodiments, stimulator 200 includes an electronic filter, such as a low-pass filter that filters out or eliminates high frequency components from the signal to smooth out the signal before it reaches the electrodes 204, 206. The low-pass filter may comprise a digital or analog filter or simply a capacitor placed in series between the signal generator and the electrode/interface. When the signal is generated, energy switching and electrical noise typically add unwanted high frequency spikes back into the signal. In addition, the pulsing of the sinusoidal bursts may induce high frequency components in the signal. By filtering the signal just before it reaches the electrodes, a smoother, cleaner signal is applied to the individual, thereby reducing the pain and discomfort felt by the individual and allowing a higher amplitude to be applied to the individual. This allows a sufficiently strong signal to be applied to reach a deeper nerve, such as the vagus nerve, without causing too much pain and discomfort to the individual at the surface of their skin.

Referring now to FIGS. 3C and 3D, another embodiment of a stimulator 200a comprises a housing 202a and first and second electrodes 204a, 206a extending from one surface of housing 202a. As in the previous embodiment, housing 202a contains the electronic components, signal generator and energy source (not shown) that are used to generate the signals that drive electrical impulses through electrodes 204a, 206a.

Stimulator 200a comprises a cover 210a for protecting electrodes 204a, 206a from the external environment. Cover 210 also ensures that electrodes 204a, 206a will not contact an individual's tissue when the device is not intended to be used (e.g., in the event that the device is accidently turned ON and electric current is passed through electrodes when not in use). In certain embodiments, cover 210a is rotatably coupled to housing 202a such that it can be moved between a first position (FIG. 3D), wherein the electrodes 204a, 206a are exposed for stimulation, and a second position (FIG. 3C), wherein the electrodes are housed and protected within the cover 210. Cover 210a may comprise any suitable material, such as polyphenylene ether (PPE), plastic, or other polymers.

Housing 202a preferably includes curved side surfaces 216a, 218a with substantially planar surfaces 220a therebetween to form an overall disc-like shape that is cut off on the upper portion of the disc. A control panel 240a may be included on one of the side surfaces 216a. Control panel 240a includes a number of user controls and/or device status indicators. In alternative embodiments, the controls and status indicators are located on a separate device, such as a mobile device, that is wirelessly (e.g. Bluetooth or the like) coupled to stimulator 200a.

In a preferred embodiment, control panel 240a includes intensity controls 242a for controlling the level of intensity or amplitude of the electrical impulses generated by stimulator 200a. Intensity controls 242a extend outward from side surfaces 216a so that the user may tactically identify and control intensity controls 242a.

Control panel 240a may further comprise a battery life indicator and/or a dose duration indicator (not shown). These indicators may include, for example, LEDs or other light sources, to facilitate identification by the user.

Stimulator 200a may further include a power control 250a for turning ON the device. Power control 250a may also include an LED or other light source for illuminating power control 250a when the device has been turned ON. In one embodiment, power control 250a is located on planar surfaces 220a, although it will be understood that power control 250a may be located on control panel 240a or elsewhere on stimulator 200a.

In certain embodiments, stimulator 200a further includes a charging pad (not shown) coupled to a suitable connector for providing power to stimulator 200a and/or recharging the battery within stimulator 200a. The charging pad may comprise any suitable charging source, such as an inductive charging source that provides power via inductive transmission through the lower surface 228a of housing 202a. Stimulator 200 may further include a gel pad (not shown) that includes a conductive gel positioned to contact electrodes 204a, 206a when gel pad is positioned over upper surface 229a.

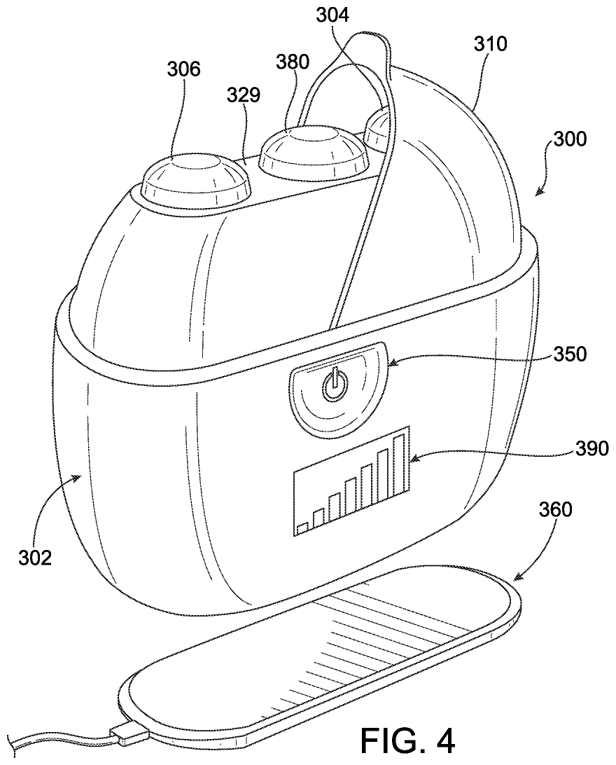

Referring now to FIG. 4, another embodiment of a stimulator 300 has a similar construction as stimulator 200 described above. In addition, stimulator 300 includes a sensor 380 extending from upper surface 329 of housing 302. Sensor 380 is preferably located between electrodes 304, 306, although it will be recognized that sensor 380 may be positioned in other locations on housing 302. For example, sensor 380 may be positioned on one of the side surfaces of housing 202, on the bottom surface 328 of housing, or electrodes 304, 306 may be positioned closer together such that sensor 380 is positioned on either side of electrodes 304, 306.

In certain embodiments, sensor 380 comprises a heart pulse sensor that detects the heart pulse of the individual when the sensor 380 is placed in contact with, or near, the outer skin surface of the individual. As discussed above, the heart pulse sensor detects that the sensor is close to, or adjacent, a source of heart pulse, such as the carotid artery in the individual's neck or the radial artery in the wrist. The heart pulse sensor may be any suitable sensor known in the art, for detecting the heart pulse of an individual, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 380 is located adjacent to, or near, the individual's heart pulse.

Sensor 380 is configured to generate an output that indicates the proximity of a heart pulse in the individual. The output may be generated and transmitted via wire, wirelessly, or waveguide, to a control unit within stimulator 300, a mobile device, processor, server, or other logic or computing device. This output provides an indication that electrodes 304, 306 are positioned optimally to modulate the target nerve, e.g., the vagus nerve.

Stimulator 300 further includes a position indicator 390 coupled to sensor 380, the control until within stimulator 300, or a separate device, and configured to provide indication of the position of the stimulator relative to the heart pulse within the individual. As discussed above, position indicator is configured to generate an alert when sensor 380 has detected the target nerve. The alert may be, for example, a visual, tactile or audial alert, that provides the user with an indication that the sensor 380 has detected the target location.

In certain embodiments, position indicator 380 is configured to transmit an alert that is associated with the magnitude of the heart pulse. In other embodiments, position indicator 390 is further be configured to provide a second alert when the magnitude of the heart pulse reaches a threshold level associated with optimal positioning of the sensor 380 and/or electrodes 304, 306.

Stimulator 200 may include additional sensors, such as, for example, biosensors, feedback sensors, chemical sensors, optical sensors, acoustic sensors, vibration sensors, motion sensors, fluid sensors, radiation sensors, temperature sensors, motion sensors, proximity sensors, fluid sensors, or others. The sensors may generate an output, such as one or more outputs, which are communicated, via wire, wirelessly or waveguide, to the stimulator 200, a mobile device, processor, server, or other logic or computing device. The output may be used as an input to one or more of the foregoing devices to forecast or avert an imminent onset or predicted upcoming onset of a symptom, episode, condition or disease. For example, as disclosed in U.S. Patent App. Pub. No. 2017/0120052, which is incorporated herein by reference in its entirety for at least these purposes as if copied and pasted herein, as disclosed herein, and for all purposes as if copied and pasted herein, such as all structures, all functions, and all methods of manufacture and use, as disclosed therein.

FIG. 5 provides a more detailed view of use of stimulator 300 when positioned to stimulate the vagus nerve at the neck location. The anatomy shown in FIG. 5 is a cross-section of half of the neck at vertebra level C6. The vagus nerve 382 is identified in FIG. 5, along with the carotid sheath 384 that is identified there in bold peripheral outline. The carotid sheath encloses not only the vagus nerve, but also the internal jugular vein 386 and the common carotid artery 387. Structures that may be identified near the surface of the neck include the external jugular vein 388 and the sternocleidomastoid muscle 389, which protrudes when the individual turns his or her head. Additional organs in the vicinity of the vagus nerve include the trachea 392, thyroid gland 393, esophagus 394, scalenus anterior muscle 395, scalenus medius muscle 396, levator scapulae muscle 397, splenius colli muscle 398, semispinalis capitis muscle 399, semispinalis colli muscle 401, longus colli muscle and longus capitis muscle 402. The sixth cervical vertebra 403 is shown with bony structure indicated by hatching marks.

In use, upper surface 399 of stimulator 300 is positioned near the outer skin surface 405 of the neck of the individual such that electrodes 304, 306 are in contact with surface 405. In certain embodiments, sensor 380 (not shown in FIG. 5) will also be in contact with skin surface 405. The user turns the device ON and moves the stimulator 300 along skin surface 405 until sensor 380 detects the heart pulse within carotid artery 387. Once the heart pulse has been detected, stimulator 300 is in the optimal position to transmit electrical impulses through electrodes 304, 306 to vagus nerve 382.

In certain embodiments, sensor 380 detects the magnitude of the heart pulse and generates a signal associated with such magnitude. In these embodiments, the user may elect to continue to reposition stimulator 300 until the magnitude of the heart pulse reaches a threshold level. Indicator 390 may be configured to provide a second alert to the user that such position has been reached.

Stimulation may be performed on the left or right vagus nerve or on both of them simultaneously and alternately. The position and angular orientation of the device are adjusted about that location until the individual perceives stimulation when current is passed through the stimulator electrodes. The applied current is increased gradually, first to a level wherein the individual feels sensation from the stimulation. The energy is then increased, but is set to a level that is less than one at which the individual first indicates any discomfort. Straps, harnesses, or frames may be used to maintain the stimulator in position. The stimulator signal may have a frequency and other parameters that are selected to produce a therapeutic result in the individual, i.e., stimulation parameters for each individual are adjusted on an individualized basis. Ordinarily, the amplitude of the stimulation signal is set to the maximum that is comfortable for the individual, and then the other stimulation parameters are adjusted.

The stimulation is then performed with a sinusoidal burst waveform like that shown in FIG. 2. As seen there, individual sinusoidal pulses have a period of τ, and a burst consists of N such pulses. This is followed by a period with no signal (the inter-burst period). The pattern of a burst followed by silent inter-burst period repeats itself with a period of T. For example, the sinusoidal period τ may be between about 50-1000 microseconds and a frequency of about 1-20 kHz, preferably between about 100-400 microseconds and a frequency of about 2.5-10 kHz, more preferably about 133-400 microseconds and a frequency of about 2.5-7.5 kHz and even more preferably about 200 microseconds and a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2C to make the bursts discernable). When these example values are used for T and τ, the waveform contains significant Fourier components at higher frequencies ( 1/200 microseconds=5000/sec), as compared with those contained in transcutaneous nerve stimulation waveforms.

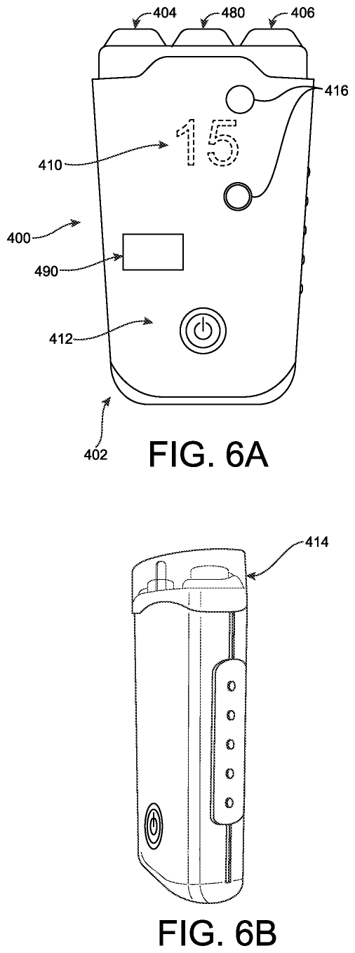

Referring now to FIGS. 6A and 6B, another embodiment of a stimulator 400 that includes a housing 402, a display 410, electrodes 404, 406, a power button 412 a cap 414 and a control button 416. In some embodiments, the neurostimulator 400 includes a speaker housed via the housing 400 and powered via the battery. In some embodiments, the neurostimulator 400 includes a microphone housed via the housing 402 and powered via the battery. The housing 402 houses a signal generator and a battery. The housing 402 is opaque, but can be transparent. The battery powers the signal generator and the display. The power button 408 turns the neurostimulator 400 on and off. The button 408 can be a mechanical button or a touch-enabled surface, which can be haptic or configured to receive a touch input, a slide input, a gesture input, or others. The electrodes 404, 406 contact a skin of an individual and conduct a stimulation energy, such as an electrical current, an electrical impulse, an actuation, or others, from the signal generator to the skin of the individual.

The display 410, which can present in monochrome, grayscale, or color, indicates a status of the neurostimulator 400, such as on, off, charging, dosage amount total, dosage amount remaining, stimulation time total, stimulation time remaining, or others. The display 410 can be of any type, such as a segment display, a liquid crystal display (LCD), an electrophoretic display, a field emission display (FED), or others, whether rigid, elastic, resilient, bendable, or flexible. The display 410 can be configured to receive a touch-input, including a gesture, a slide, or others.

The cap 414 is mounted to the housing 402, such as via snug fit, friction, fastening, mating, adhering, or others. The cap 414 is transparent, but can be opaque. The cap 414 covers and protects the electrodes 404, 406 from mechanical damage, interference, moisture, or others. The control button(s) 416 are operably coupled to the signal generator and is thereby configured to increase or decrease an intensity of the stimulation by controlling the signal generator. The control button(s) 416 can be a mechanical buttons or a touch-enabled surfaces, which can be haptic or configured to receive a touch input, a slide input, a gesture input, or others. The neurostimulator 400 can be charged via a charging station (not shown), whether in a wired, wireless, or waveguide manner.

Stimulator 400 further includes a sensor 480 preferably located between electrodes 404, 406, although it will be recognized that sensor 480 may be positioned in other locations on housing 402. For example, sensor 480 may be positioned on one of the side surfaces of housing 402, on the bottom surface of housing 402, or electrodes 404, 406 may be positioned closer together such that sensor 480 is positioned on either side of electrodes 404, 406.

In certain embodiments, sensor 480 comprises a heart pulse sensor that detects the heart pulse of the individual when the sensor 480 is placed in contact with, or near, the outer skin surface of the individual. As discussed above, the heart pulse sensor detects that the sensor is close to, or adjacent, a source of heart pulse, such as the carotid artery in the individual's neck or the radial artery in the wrist.

Stimulator 400 further includes a position indicator 490 coupled to sensor 480, the control until within stimulator 400, or a separate device, and configured to provide indication of the position of the stimulator relative to the heart pulse within the individual. As discussed above, position indicator is configured to generate an alert when sensor 480 has detected the target nerve. The alert may be, for example, a visual, tactile or audial alert, that provides the user with an indication that the sensor 380 has detected the target location.

Neurostimulator 400 can be a multi-use, hand-held, rechargeable, portable device comprising of a rechargeable battery, a set of signal-generating and amplifying electronics, and a control button for operator control of a signal amplitude. The device provides visible (display) and audible (beep) feedback on the device and stimulation status. A pair of stainless steel surfaces, which are a set of skin contact surfaces, allows a delivery of an electrical signal. The individual applies an electrode gel to the contact surfaces to maintain an uninterrupted conductive path from the contact surfaces to the skin on the neck of the individual. The stimulation surfaces are capped when not in use. The neurostimulator 400 can produce a low voltage electric signal including about five 5,000 Hz electric pulses (or less or more) that are repeated at a rate of 25 Hz (or less or more). A waveform of the electric pulses is approximately a sine wave with a peak voltage limited to about 24 volts (or less or more) when placed on the skin of the neck of the individual and a maximum output current of 60 mA (or less or more). The signal is transmitted through the skin of the neck to the vagus nerve. The neurostimulator 400 allows the individual to appropriately position and adjust a stimulation intensity as instructed a healthcare provider. Further details of appropriate waveforms and electrical signals and how to generate and transmit such signals to a desired nerve can be found in U.S. Pat. Nos. 8,874,205; 9,333,347; 9,174,066; 8,914,122 and 9,566,426, which are incorporated herein in their entireties by reference for at least these purposes as if copied and pasted herein, as disclosed herein, and for all purposes as if copied and pasted herein, such as all structures, all functions, and all methods of manufacture and use, as disclosed therein. Each dose can be applied for two minutes, after which the neurostimulator automatically stops delivering the neurostimulation. The neurostimulator 400 can allow for single or multiple uses or sessions. The neurostimulator can deliver a fixed number of treatments within a 24-hour period (or less or more). Once a maximum daily number of treatments has been reached, the neurostimulator 400 will not deliver any more treatments until a following 24-hour period expires. The neurostimulator can be charged via a charging station. The neurostimulator can allow for a fixed number of treatments within a defined time period, such as thirty one days or ninety three days, or some other period of time. A more complete description of systems for initially provisioning and refilling stimulator 400 can be found in U.S. patent application Ser. No. 16/229,299, filed Dec. 22, 2017, the complete disclosure of which is incorporated herein by reference for all purposes.

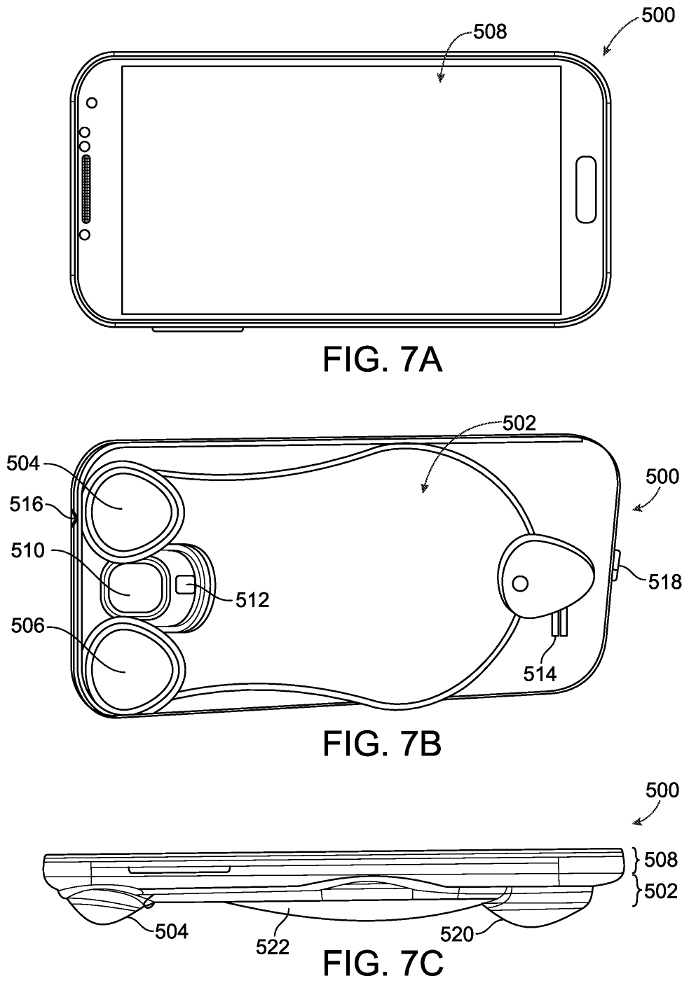

Another embodiment of an electrode-based stimulator 500 is shown in FIGS. 7A-7C. As shown, the stimulator comprises a smartphone with its back cover removed and and joined to a housing 502 that comprises a pair of electrode surfaces 504, 506 along with circuitry to control and energy the electrodes and interconnect with the smartphone. FIG. 7A shows the side of the smartphone 508 with a touch-screen. FIG. 7B shows the housing of the stimulator 502 joined to the back of the smartphone. Portions of the housing lie flush with the back of the smartphone, with windows to accommodate smartphone components that are found on the original back of the smartphone. Such components may also be used with the stimulator, e.g., the smartphone's rear camera 510, flash 512 and speaker 514. Other original components of the smartphone may also be used, such as the audio headset jack socket 516 and multi-purpose jack 518. Note that the original components of the smartphone shown in FIGS. 7A-7C correspond to a Samsung Galaxy smartphone, and their locations may be different for embodiments that use different smartphone models by different smartphone manufacturers. Note that tablets can be used as well.

FIG. 7C shows that several portions of the housing 502 protrude towards the back. The two electrode surfaces 504, 506 protrude so that they may be applied to the skin of the individual. The stimulator may be held in place by straps or frames or collars, or the stimulator may be held against the individual's body by hand. In some embodiments, the neurostimulator may comprise a single such electrode surface or more than two electrode surfaces.

A dome 520 also protrudes from the housing, so as to allow the device to lie more or less flat on a table when supported also by the electrode surfaces. The dome also accommodates a relatively tall component that may lie underneath it, such as a battery. Alternatively, the stimulation device may be energed by the smartphone's battery. The belly 522 of the housing protrudes to a lesser extent than the electrodes and dome. The belly accommodates a printed circuit board that contains electronic components within the housing (not shown), as described below.

Stimulator 500 may also comprise a position sensor (not shown), such as one of the sensors describe above. The position sensor may, for example, be located in dome 520, or belly 522 of the housing. A more complete description of a stimulator for use with a mobile device can be found in commonly-assigned U.S. Pat. No. 9,375,571, the complete disclosure of which is incorporated herein by reference for all purposes.

Electronics and Software of the Stimulator

In some embodiments, the signal waveform (FIG. 2) that is to be applied to electrodes of the stimulator is initially generated in a component of an impulse generator that is exterior to, and remote from, the mobile phone housing. The mobile phone preferably includes a software application that can be downloaded (e.g., mobile app store, USB cable, memory stick, Bluetooth connection) into the phone to receive, from the external control component, a wirelessly transmitted waveform, or to receive a waveform that is transmitted by cable, e.g., via a multi-purpose jack. If the waveforms are transmitted in compressed form, they are preferably compressed in a lossless manner, e.g., making use of FLAC (Free Lossless Audio Codec). Alternatively, the downloaded software application may itself be coded to generate a particular waveform that is to be applied to the electrodes and subsequently conveyed to the external interface of the electrode assembly. In some embodiments, the software application is not downloaded from outside the device, but is instead available internally, for example, within read-only-memory that is present within the housing of the stimulator.

In some embodiments, the waveform is first conveyed by the software application to contacts within the phone's speaker output or the earphone jack socket, as though the waveform signal were a generic audio waveform. That pseudo-audio waveform will generally be a stereo waveform, representing signals that are to be applied to the “left” and “right” electrodes. The waveform will then be conveyed to the housing of the stimulator. as follows. The housing of the stimulator may have an attached dangling audio jack that is plugged into the speaker output or the earphone jack socket whenever electrical stimulation is to be performed, or the electrical connection between the contacts of the speaker output or the earphone jack socket and the housing of the stimulator may be hard-wired. In either case, electrical circuits on a printed circuit board located under the belly of the housing of the stimulator may then shape, filter, and/or amplify the pseudo-audio signal that is received via the speaker output or earphone jack socket. An energy amplifier within the housing of the stimulator may then drive the signal onto the electrodes, in a fashion that is analogous to the use of an audio energy amplifier to drive loudspeakers. Alternatively, the signal processing and amplification may be implemented in a separate device that can be plugged into sockets on the phone and/or housing of the stimulator, to couple the software application and the electrodes.

In addition to passing the stimulation waveform from the smartphone to the stimulator housing as described herein, the smartphone may also pass control signals to the stimulator housing. Thus, the stimulation waveform may generally be regarded as a type of analog, pseudo-audio signal, but if the signal contains a signature series of pulses signifying that a digital control signal is about to be sent, logic circuitry in the stimulator housing may then be set to decode the series of digital pulses that follows the signature series of pulses, analogous to the operation of a modem.

Many of the steps that direct the waveform to the electrodes, including steps that may be controlled by the user via the touchscreen, are implemented in the above-mentioned software application. By way of example, the software application may be written for a phone that uses the Android operating system. Such applications are typically developed in the Java programming language using the Android Software Development Kit (SDK), in an integrated development environment (IDE), such as Eclipse [Mike WOLFSON. Android Developer Tools Essentials. Sebastopol, California: O'Reilly Media Inc., 2013; Ronan SCHWARZ, Phil Duston, James Steele, and Nelson To. The Android Developer's Cookbook. Building Applications with the Android SDK, Second Edition. Upper Saddle River, NJ: Addison-Wesley, 2013, the disclosure of which is incorporated herein by reference for all purposes as if copied and pasted herein; Shane CONDER and Lauren Darcey. Android Wireless Application Development, Second Edition. Upper Saddle River, NJ: Addison-Wesley, 2011; Jerome F. DIMARZIO. Android—A Programmer's Guide. New York: McGraw-Hill. 2008. pp. 1-319, the disclosure of which is incorporated herein by reference for all purposes as if copied and pasted herein]. Application programming interfaces (APIs) that are particularly relevant to the audio features of such an Android software application (e.g., MediaPlayer APIs) are described by: Android Open Source Project of the Open Handset Alliance. Media Playback, at web domain developer.android.com with subdomain/guide/topics/media/, Jul. 18, 2014, the disclosure of which is incorporated herein by reference for all purposes as if copied and pasted herein. Those APIs can be relevant to a use of the smartphone camera capabilities, as described below. Additional components of the software application are available from device manufacturers [Samsung Mobile SDK, at web domain developer.samsung.com with subdomain/samsung-mobile-sdk, Jul. 18, 2014, the disclosure of which is incorporated herein by reference for all purposes as if copied and pasted herein].

In some embodiments, the stimulator and/or smartphone will include a user control, such as a switch or button, that disables/enables the stimulator. Preferably, the switch will automatically disable some, many, most, or all smartphone functions when the stimulator is enabled (and vice versa). This ensures that the medical device functionality of the smartphone is completely segregated from the rest of the phone's functionality. In some embodiments, the switch will be password-controlled such that only the individual/owner of the stimulator/phone will be able to enable the stimulator functionality. In one such embodiment, the switch will be controlled by a biometric scan (e.g., fingerprint, optical scan or the like) such that the stimulator functionality can only be used by the individual. This ensures that only the individual will be able to use the prescribed therapy in the event the phone is lost or stolen.

The stimulator and/or phone can also include software that allows the individual to order more therapy doses over the internet (discussed in more detail below in connection with the docking station). The purchase of such therapy doses will require physician authorization through a prescription or the like. To that end, the software can include an authorization code for entry in order for the individual to download authorization for more therapies. In some embodiments, without such authorization, the stimulator will be disabled and will not deliver therapy.

Although the device shown in FIGS. 7A-7C is an adapted commercially available smartphone, it is understood that in some embodiments, the housing of the stimulator may also be joined to and/or energized by a wireless device that is not a phone (e.g., Wi-Fi enabled device, wearable, tablet). Alternatively, the stimulator may be coupled to a phone or other Wi-Fi enabled device through a wireless connection for exchanging data at short distances, such as Bluetooth or the like. In this embodiment, the stimulator housing is not attached to the smartphone and, therefore, may comprise a variety of other shapes and sizes that are convenient for the individual to carry in his or her purse, wallet or pocket.

In some embodiments, the stimulator housing may be designed as part of a protective or decorative case for the phone that can be attached to the phone, similar to standard phone cases. In one such embodiment, the stimulator/case may also include additional battery life for the phone and may include an electrical connection to the phone's battery to recharge the battery (e.g., part of a Mophie® or the like). This electrical connection may also be used to couple the smartphone to the stimulator.

Embodiments with Distributed Controllers

In some embodiments, significant portions of the control of the vagus nerve stimulation reside in controller components that are physically separate from the housing of the stimulator. In these embodiment, separate components of the controller and stimulator housing generally communicate with one another wirelessly, although wired or waveguide communication is possible. Thus, the use of wireless technology avoids the inconvenience and distance limitations of interconnecting cables.

First, the stimulator may be constructed with the minimum number of components needed to generate the stimulation pulses, with the remaining components placed in parts of the controller that reside outside the stimulator housing, resulting in a lighter and smaller stimulator housing. In fact, the stimulator housing may be made so small that it could be difficult to place, on the stimulator housing's exterior, switches and knobs that are large enough to be operated easily. Instead, the user may generally operate the device using the smartphone touchscreen.

Second, the controller 130 may be given additional functions when free from the limitation of being situated within or near the stimulator housing. For example, one may add to the controller a data logging component that records when and how stimulation has been applied to the individual, for purposes of medical recordkeeping and billing. The complete electronic medical record database for the individual may be located far from the stimulator (e.g., somewhere on the internet), and the billing system for the stimulation services that are provided may also be elsewhere, so it would be useful to integrate the controller into that recordkeeping and billing system, using a communication system that includes access to the internet or telephone networks.

Third, communication from the databases to the controller would also be useful for purposes of metering electrical stimulation of the individual, when the stimulation is self-administered. For example, if the prescription for the individual only permits only a specified amount of stimulation energy to be delivered during a single session of vagus nerve stimulation, followed by a wait-time before allowing the next stimulation, the controller can query the database and then permit the stimulation only when the prescribed wait-time has passed. Similarly, the controller can query the billing system to assure that the individual's account is in order, and withhold the stimulation if there is a problem with the account.

Fourth, as a corollary of the previous considerations, the controller may be constructed to include a computer program separate from the stimulating device, in which the databases are accessed via cell phone or internet connections.

Fifth, in some applications, it may be desired that the stimulator housing and parts of the controller be physically separate. For example, when the individual is a child, one wants to make it impossible for the child to control or adjust the vagus nerve stimulation. The best arrangement in that case is for the stimulator housing to have no touchscreen elements, control switches or adjustment knobs that could be activated by the child. Alternatively, any touchscreen elements, switches and knobs on the stimulator can be disabled, and control of the stimulation then resides only in a remote controller with a child-proof operation, which would be maintained under the control of a parent or healthcare provider.

Sixth, in some applications, the particular control signal that is transmitted to the stimulator by the controller will depend on physiological and environmental signals that are themselves transmitted to and analyzed by the controller. In such applications, many of the physiological and environmental signals may already be transmitted wirelessly, in which case it is most convenient to design an external part of the controller as the hub of all such wireless activity, including any wireless signals that are sent to and from the stimulator housing.

With these considerations in mind, an embodiment of can include a mobile device that may send/receive data to/from the stimulator, and may send/receive data to/from databases and other components of the system, including those that are accessible via the internet (or another network such as local area, wide area, satellite, cellular). Typically, the mobile device will be a laptop computer attached to additional components needed for it to accomplish its function. Thus, prior to any particular stimulation session, the mobile device may load into the stimulator parameters of the session, including waveform parameters, or the actual waveform.

In some embodiments, the mobile device is also used to limit the amount of stimulation energy that may be consumed by the individual during the session, by charging the stimulator's rechargable battery with only a specified amount of releasable electrical energy, which is different than setting a parameter to restrict the duration of a stimulation session. Thus, the mobile device may comprise a energy supply that may be connected to the stimulator's rechargable battery, and the mobile device meters the recharge. As a practical matter, the stimulator may therefore use two batteries, one for applying stimulation energy to the electrodes (the charge of which may be limited by the mobile device) and the other for performing other functions. Methods for evaluating a battery's charge or releasable energy can be as disclosed in U.S. Pat. No. 7,751,891, entitled Energy supply monitoring for an implantable device, to ARMSTRONG et al, the disclosure of which is incorporated herein by reference for all purposes as if copied and pasted herein. Alternatively, some control components within the stimulator housing may monitor the amount of electrode stimulation energy that has been consumed during a stimulation session and stop the stimulation session when a limit has been reached, irrespective of the time when the limit has been reached.

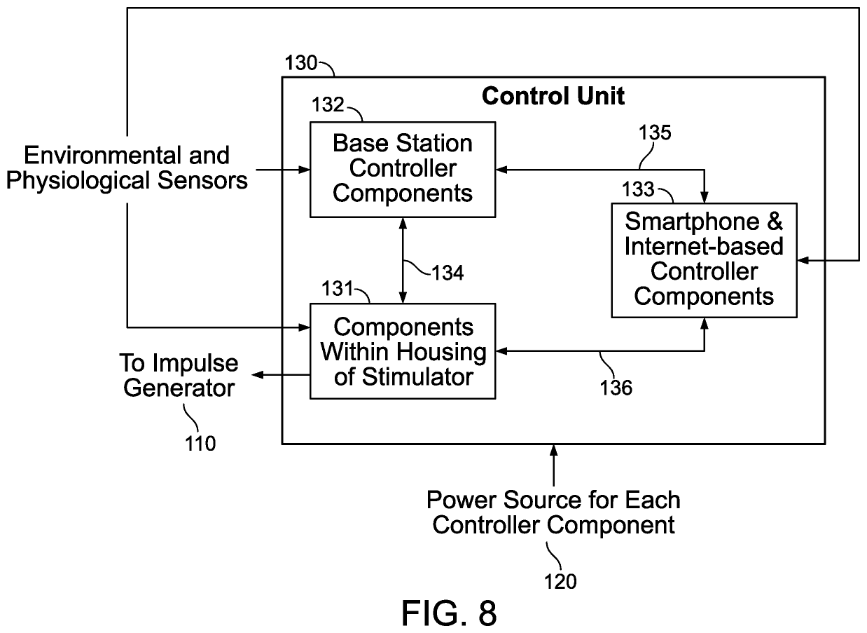

The communication connections between different components of the stimulator's controller are shown in FIG. 8, which is an expanded representation of the control unit 130 in FIG. 1. Connection between the mobile device controller components 132 and components within the stimulator housing 131 is denoted in FIG. 8 as 134. Connection between the mobile device controller components 132 and internet-based (or network based) or smartphone components 133 is denoted as 135. Connection between the components within the stimulator housing 331 and internet-based or smartphone components 133 is denoted as 136. For example, control connections between the smartphone and stimulator housing via the audio jack socket would fall under this category, as would any wireless communication directly between the stimulator housing itself and a device situated on the internet. In principle, the connections 134, 135 and 136 in FIG. 8 may be either wired or wireless or waveguide-based. Different embodiments may lack one or more of the connections.

Although infrared or ultrasound wireless control might be used to communicate between components of the controller, they are not preferred because of line-of-sight limitations. Instead, the communication between devices preferably makes use of radio communication within unlicensed ISM frequency bands (260-470 MHz, 902-928 MHz, 2400-2.4835 GHz). Components of the radio frequency system in devices in 331, 332, and 333 typically comprise a system-on-chip transceiver with an integrated microcontroller; a crystal; associated balun & matching circuitry, and an antenna [Dag GRINI. RF Basics, RF for Non-RF Engineers. Texas Instruments, Post Office Box 655303, Dallas, Texas 75265, 2006, the disclosure of which is incorporated herein by reference for all purposes as if copied and pasted herein].

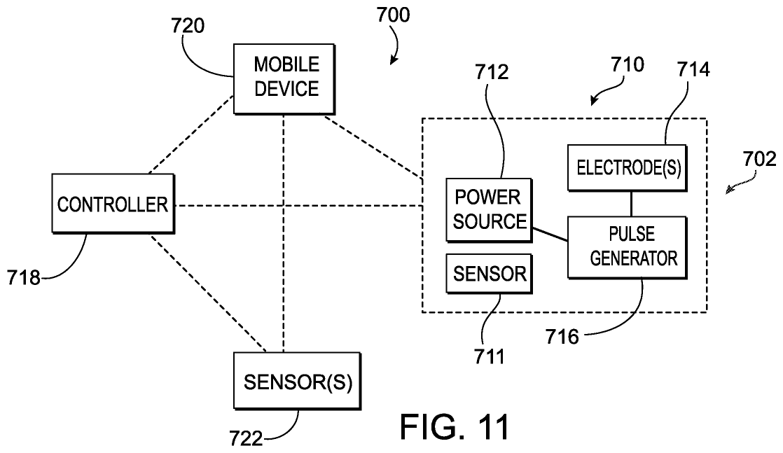

Transceivers based on 2.4 GHz offer high data rates (greater than 1 Mbps) and a smaller antenna than those operating at lower frequencies, which makes them suitable for with short-range devices. Furthermore, a 2.4 GHz wireless standard (e.g., Bluetooth, Wi-Fi, and ZigBee) may be used as the protocol for transmission between devices. Although the ZigBee wireless standard operates at 2.4 GHz in most jurisdictions worldwide, it also operates in the ISM frequencies 868 MHz in Europe, and 915 MHz in the USA and Australia. Data transmission rates vary from 20 to 250 kilobits/second with that standard. Because many commercially available health-related sensors may operate using ZigBee, its use may be recommended for applications in which the controller uses feedback and feedforward methods to adjust the individual's vagus nerve stimulation based on the sensors' values, as described below in connection with FIG. 11 [ZigBee Wireless Sensor Applications for Health, Wellness and Fitness. ZigBee Alliance 2400 Camino Ramon Suite 375 San Ramon, CA 94583].

A 2.4 GHz radio has higher energy consumption than radios operating at lower frequencies, due to reduced circuit efficiencies. Furthermore, the 2.4 GHz spectrum is crowded and subject to significant interference from microwave ovens, cordless phones, 802.11b/g wireless local area networks, Bluetooth devices, etc. Sub-GHz radios enable lower energy consumption and can operate for years on a single battery. These factors, combined with lower system cost, make sub-GHz transceivers ideal for low data rate applications that need maximum range and multi-year operating life.

The antenna length needed for operating at different frequencies is 17.3 cm at 433 MHz, 8.2 cm at 915 MHz, and 3 cm at 2.4 GHz. Therefore, unless the antenna is included in a neck collar that supports the device shown in FIG. 3, the antenna length may be a disadvantage for 433 MHz transmission. The 2.4 GHz band has the advantage of enabling one device to serve in all major markets worldwide since the 2.4 GHz band is a global spectrum standard. However, 433 MHz is a viable alternative to 2.4 GHz for most of the world, and designs based on 868 and 915 MHz radios can serve the US and European markets with a single product.

Range is determined by the sensitivity of the transceiver and its output energy. A primary factor affecting radio sensitivity is the data rate. Higher data rates reduce sensitivity, leading to a need for higher output energy to achieve sufficient range. For many applications that require only a low data rate, the preferred rate is 40 Kbps where the transceiver can still use a standard off-the-shelf 20 parts per million crystal.

A signal waveform that might be transmitted wirelessly to the stimulator housing was shown in FIGS. 2B and 2C. As seen there, individual sinusoidal pulses have a period of tau, and a burst consists of N such pulses. This is followed by a period with no signal (the inter-burst period). The pattern of a burst followed by silent inter-burst period repeats itself with a period of T. For example, the sinusoidal period tau may be 200 microseconds; the number of pulses per burst may be N=5; and the whole pattern of burst followed by silent inter-burst period may have a period of T=40000 microseconds, which is comparable to 25 Hz stimulation (a much smaller value of T is shown in FIG. 2C to make the bursts discernable). When these exemplary values are used for T and tau, the waveform contains significant Fourier components at higher frequencies ( 1/200 microseconds=5000/sec). Such a signal may be easily transmitted using 40 Kbps radio transmission. Compression of the signal is also possible, by transmitting only the signal parameters tau, N, T, Emax, etc., but in that case the stimulator housing's control electronics would then have to construct the waveform from the transmitted parameters, which would add to the complexity of components of the stimulator housing.

However, because it is contemplated that sensors attached to the stimulator housing may also be transmitting information, the data transfer requirements may be substantially greater than what is required only to transmit the signal shown in FIG. 2. Therefore, the devices and methods disclosed herein may make use of any frequency band, not limited to the ISM frequency bands, as well as techniques known in the art to suppress or avoid noise and interferences in radio transmission, such as frequency hopping and direct sequence spread spectrum.

When an individual is using the stimulation device to perform self-stimulation therapy, e.g., at home or at a workplace, he or she will follow the steps that are now described. It is assumed that the optimal stimulation position has already been marked on the individual's neck, as described above and that a reference image of the fluorescent spots has already been acquired. The previous stimulation session will ordinarily have discharged the rechargeable batteries of the stimulator housing, and between sessions, the mobile device will have been used to recharge the stimulator at most only up to a minimum level. If the stimulator's batteries had charge remaining from the previous stimulation session, the mobile device will discharge the stimulator to a minimum level that will not support stimulation of the individual.

The individual can initiate the stimulation session using the mobile phone or mobile device (e.g., laptop computer) by invoking a computer program (on the laptop computer or through an app on the mobile phone) that is designed to initiate use of the stimulator. The programs in the smartphone and mobile device may initiate and interact with one another wirelessly, so in what follows, reference to the program (app) in the smartphone may also apply to the program in the mobile device, because both may be operating in tandem. For security reasons, the program would begin with the request for a user name and a password, and that user's demographic information and any data from previous stimulator experiences would already be associated with it in the login account. The smartphone may also be used to authenticate the individual using a fingerprint or voice recognition app, or other reliable authentication methods. If the individual's physician has not authorized further treatments, the mobile device will not charge the stimulator's batteries, and instead, the computer program will call or otherwise communicate with the physician's computer requesting authorization. After authorization by the physician is received, the computer program (on the laptop computer or through an app on the mobile phone) may also query a database that is ordinarily located somewhere on the internet to verify that the individual's account is in order. If it is not in order, the program may then request prepayment for one or more stimulation sessions, which would be paid by the individual using a credit card, debit card, PayPal, cryptocurrency, bitcoin, or the like. The computer program will also query its internal database or that of the mobile device to determine that sufficient time has elapsed between when the stimulator was last used and the present time, to verify that any required wait-time has elapsed.

Having received authorization to perform a nerve stimulation session, the individual interface computer program will then ask the individual questions that are relevant to the selection of parameters that the mobile device will use to make the stimulator ready for the stimulation session. The questions that the computer program asks are dependent on the condition for which the individual is being treated, which for present purposes is considered to be treatment for an autoimmune disease or disorder. The questions may be things like (1) is this an acute or prophylactic treatment? (2) if acute, then how severe is your pain and in what locations, how long have you had it, (3) has anything unusual or noteworthy occurred since the last stimulation? etc.

Having received such preliminary information from the individual, the computer programs will perform instrument diagnostic tests and make the stimulator ready for the stimulation session. In general, the algorithm for setting the stimulator parameters will have been decided by the physician and will include the extent to which the stimulator batteries should be charged, which the vagus nerve should be stimulated (right or left), and the time that the individual should wait after the stimulation session is ended until initiation of a subsequent stimulation session. The computer will query the physician's computer to ascertain whether there have been any updates to the algorithm, and if not, will use the existing algorithm. The individual will also be advised of the stimulation session parameter values by the interface computer program, so as to know what to expect.

Once the mobile device has been used to charge the stimulator's batteries to the requisite charge, the computer program (or smartphone app) will indicate to the individual that the stimulator is ready for use. At that point, the individual would clean the electrode surfaces, and make any other preliminary adjustments to the hardware. The stimulation parameters for the session will be displayed, and any options that the individual is allowed to select may be made. Once the individual is ready to begin, he or she will press a “start” button on the touchscreen and may begin the vagus nerve stimulation.

Multiple methods may be used to test whether the individual is properly attempting to stimulate the vagus nerve (or another nerve or organ or muscle or bone) on the intended side of the neck (or another portion of a human body). For example, accelerometers and gyroscopes within the smartphone may be used to determine the position and orientation of the smartphone's touch screen relative to the individual's expected view of the screen, and a decision by the stimulator's computer program as to which hand is being used to hold the stimulator may be made by measuring capacitance on the outside of the stimulator body, which may distinguish fingers wrapped around the device versus the ball of a thumb [Raphael WIMMER and Sebastian Boring. HandSense: discriminating different ways of grasping and holding a tangible user interface. Proceedings of the 3rd International Conference on Tangible and Embedded Interaction, pp. 359-362. ACM New York, NY, 2009, the disclosure of which is incorporated herein by reference for all purposes as if copied and pasted herein]. Pressing of the electrodes against the skin will result in a resistance drop across the electrodes, which can initiate operation of the rear camera. A fluorescent image should appear on the smartphone screen only if the device is applied to the side of the neck in the vicinity of the fluorescent spots that had been applied as a tattoo earlier. If the totality of these data indicates to the computer program that the individual is attempting to stimulate the wrong vagus nerve or that the device is being held improperly, the stimulation will be withheld, and the stimulator may then communicate with the individual via the interface computer program (in the mobile phone or laptop computer) to alert the individual of that fact. T

Before logging off of the interface computer program, the individual may also review database records and summaries about all previous treatment sessions, so as to make his or her own judgment about treatment progress. If the stimulation was part of a prophylactic treatment regimen that was prescribed by the individual's physician, the individual interface computer program will remind the individual about the schedule for the upcoming self-treatment sessions and allow for a rescheduling if necessary.

For some individuals, the stimulation may be performed for as little as 60 seconds, but it may also be for up to 30 minutes or longer. The treatment is generally performed once or twice daily or several times a week, for 12 weeks or longer before a decision is made as to whether to continue the treatment. For individuals experiencing intermittent symptoms, the treatment may be performed only when the individual is symptomatic. However, it is understood that parameters of the stimulation protocol may be varied in response to heterogeneity in the pathophysiology of individuals. Different stimulation parameters may also be used as the course of the individual's condition changes.

Applications of Stimulators to the Individual

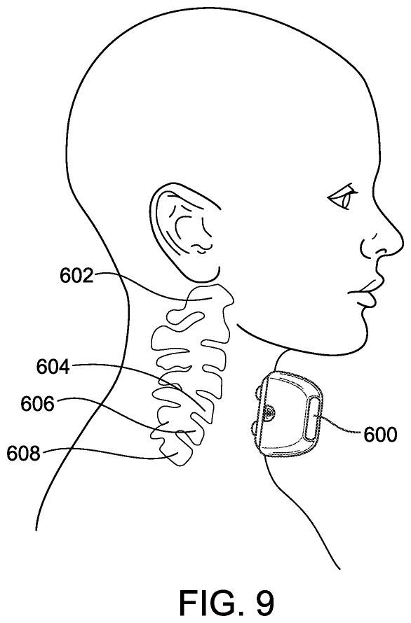

Selected nerve fibers are stimulated in different embodiments of methods that make use of the disclosed electrical stimulation devices, including stimulation of the vagus nerve at a location in the individual's neck. FIG. 9 illustrates use of a stimulator 600 to stimulate the vagus nerve at that location in the neck, in which the stimulator device 600 is shown to be applied to the target location on the individual's neck as described herein. For reference, FIG. 9 shows the locations of the following vertebrae: first cervical vertebra 602, the fifth cervical vertebra 604, the sixth cervical vertebra 606, and the seventh cervical vertebra 608.

Of course, it will be recognized that the vagus nerve may be stimulated through other mechanisms. For example, auricular vagal nerve stimulation involves stimulation of the auricular branch of the vagus nerve, often termed the Alderman's nerve or Arnold's nerve. This nerve may be stimulated through the transcutaneous systems and methods described herein by transmitting electrical impulses through the outer skin surface of the individual's ear to the auricular branch of the vagus nerve.

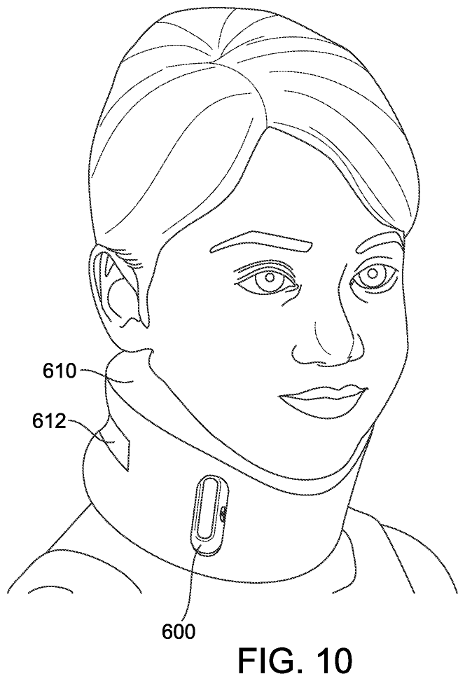

FIG. 10 shows the stimulator 600 applied to the neck of a child, which is partially immobilized with a foam cervical collar 610 that is similar to ones used for neck injuries and neck pain. The collar is tightened with a strap 612, and the stimulator is inserted through a hole in the collar to reach the child's neck surface. In such applications, the stimulator may be turned on and off remotely, using a wireless controller that may be used to adjust the stimulation parameters of the controller (e.g., on/off, stimulation amplitude, frequency, etc.).

Systems of the Present Disclosure

Referring now to FIG. 11, a system 700 for stimulating a nerve in an individual, such as the vagus nerve, includes a stimulator 712, which may include one or more electrodes 714, a pulse generator 716 and an energy source 712. Stimulator 700 may also include one or more sensors 711, such as the position sensors described above. Electrodes 714, sensors 711, pulse generator 716 and energy source 812 may all be housed in a single housing, as described in detail above. In an alternative embodiment, electrodes 714 and/or sensors 711 are disposed separately from energy source 712 and pulse generator 716. Electrodes 714 and/or sensors 711 may be coupled to these components via wired connections or wirelessly. In the latter configuration, electrodes 714 and/or sensors 711 may include suitable electronic components coupled thereto to receive the electrical impulse(s) from pulse generator 716 and to apply those electrical impulse(s) through electrodes 714 to the individual. Such electronic components may include, for example, a wireless receiver or similar component that receives the signal from a wireless transmitter coupled to pulse generator 716.

In still another embodiment, pulse generator 716 and energy source 712 are coupled to each other, either wirelessly, via wired connections, or directly in a housing that contains both components. This housing may, for example, include a wireless transmitter and may be worn by the individual in manners known to those skilled in the art, so that the signal can be transmitted from the housing to electrodes 714.

System 700 further includes a controller 718 that is coupled to stimulator 702 and may be used to select or set parameters for the stimulation protocol (amplitude, frequency, pulse width, burst number, electrode positioning etc.), the treatment regimen discussed above (i.e., duration and number of doses, etc.) or alert the individual as to the need to use or adjust the stimulator (i.e., an alarm). Controller 718 may be directly coupled to stimulator 702 via wired connectors or within the same housing, or it may be wirelessly coupled to stimulator 702.

Significant portions of the control of the vagus nerve stimulation may reside in controller components that are physically separate from stimulator 702. In this embodiment, separate components of the controller 718 and stimulator 702 generally communicate with one another wirelessly. Thus, the use of wireless technology avoids the inconvenience and distance limitations of interconnecting cables.

In certain embodiments, system 700 may further include a mobile device 720 that either couples controller 718 to stimulator 702 or vice versa. Mobile device 720 may comprise a mobile phone, such as a smartphone, a smartwatch, iPad, laptop computer or any other mobile device having a computing function and wireless transmission technology.

In addition to position sensors 711, system 700 may further include one or more additional sensors (not shown) used for detecting certain physiological parameters of the individual based on the stimulation of the nerve. The preferred sensors will include ones ordinarily used for ambulatory monitoring. For example, the sensors may comprise those used in conventional Holter and bedside monitoring applications, for monitoring heart rate and variability, ECG, respiration depth and rate, core temperature, hydration, blood pressure, brain function, oxygenation, skin impedance, and skin temperature. The sensors may be embedded in garments or placed in sports wristwatches, as currently used in programs that monitor the physiological status of soldiers [G. A. SHAW, A. M. Siegel, G. Zogbi, and T. P. Opar. Warfighter physiological and environmental monitoring: a study for the U.S. Army Research Institute in Environmental Medicine and the Soldier Systems Center. MIT Lincoln Laboratory, Lexington MA. 1 Nov. 2004, pp. 1-141]. The ECG sensors should be adapted to the automatic extraction and analysis of particular features of the ECG, for example, indices of P-wave morphology, as well as heart rate variability indices of parasympathetic and sympathetic tone. Measurement of respiration using noninvasive inductive plethysmography, mercury in silastic strain gauges or impedance pneumography is particularly advised, in order to account for the effects of respiration on the heart. A noninvasive accelerometer may also be included among the ambulatory sensors, in order to identify motion artifacts. An event marker may also be included in order for the individual to mark relevant circumstances and sensations.

For brain monitoring, the sensors may comprise ambulatory EEG sensors [CASSON A, Yates D, Smith S, Duncan J, Rodriguez-Villegas E. Wearable electroencephalography. What is it, why is it needed, and what does it entail? IEEE Eng Med Biol Mag. 29(3, 2010):44-56] or optical topography systems for mapping prefrontal cortex activation [Atsumori H, Kiguchi M, Obata A, Sato H, Katura T, Funane T, Maki A. Development of wearable optical topography system for mapping the prefrontal cortex activation. Rev Sci Instrum. 2009 April; 80(4):043704]. Signal processing methods, comprising not only the application of conventional linear filters to the raw EEG data, but also the nearly real-time extraction of non-linear signal features from the data, may be considered to be a part of the EEG monitoring [D. Puthankattil SUBHA, Paul K. Joseph, Rajendra Acharya U, and Choo Min Lim. EEG signal analysis: A survey. J Med Syst 34(2010):195-212]. In the present application, the features would include EEG bands (e.g., delta, theta, alpha, beta).

For any given position of the stimulator relative to the vagus nerve, it is also possible to infer the amplitude of the electric field that it produces in the vicinity of the vagus nerve. This is done by calculation or by measuring the electric field that is produced by the stimulator as a function of depth and position within a phantom that simulates the relevant bodily tissue [Francis Marion MOORE. Electrical Stimulation for pain suppression: mathematical and physical models. Thesis, School of Engineering, Cornell University, 2007; Bartosz SAWICKI, Robert Szmurlo, Przemyslaw Plonecki, Jacek Starzynski, Stanislaw Wincenciak, Andrzej Rysz. Mathematical Modelling of Vagus Nerve Stimulation. pp. 92-97 in: Krawczyk, A. Electromagnetic Field, Health and Environment: Proceedings of EHE '07. Amsterdam, IOS Press, 2008]. Thus, in order to compensate for movement, the controller may increase or decrease the amplitude of the output from the stimulator (u) in proportion to the inferred deviation of the amplitude of the electric field in the vicinity of the vagus nerve, relative to its desired value.

Various corresponding structures, materials, acts, and equivalents of all means or step plus function elements in various claims below are intended to include any structure, material, or act for performing the function in combination with other claimed elements as specifically claimed. Various embodiments were chosen and described in order to best explain various principles of this disclosure and various practical applications thereof, and to enable others of ordinary skill in a pertinent art to understand this disclosure for various embodiments with various modifications as are suited to a particular use contemplated.

Various diagrams depicted herein are illustrative. There can be many variations to such diagrams or steps (or operations) described therein without departing from various spirits of this disclosure. For instance, various steps can be performed in a differing order or steps can be added, deleted or modified. All of these variations are considered a part of this disclosure. People skilled in an art to which this disclosure relates, both now and in future, can make various improvements and enhancements which fall within various scopes of various claims which follow.

1. A method comprising:
applying an electrical impulse to a vagus nerve within the individual according to a stimulation protocol that includes at least two doses administered each day for a plurality of days, wherein the doses each have a duration of about ninety seconds to about 3 minutes; and
wherein the stimulation protocol is sufficient to enhance a neurostructural development in the individual.

2. The method of claim 1, wherein the stimulation protocol is sufficient to increase a connectivity of neurons within the brain of the individual.

3. The method of claim 1, wherein the stimulation protocol is sufficient to increase a neuronal plasticity within the brain of the individual.

4. The method of claim 1, wherein the doses are separated by a time frame of about five to 15 minutes.

5. The method of claim 1, wherein the stimulation protocol comprises 2 to 12 treatments/day.

6. The method of claim 1, wherein the stimulation protocol is administered every day for at least 90 days.

7. The method of claim 1, wherein the stimulation protocol is administered every day for at least one year.

8. The method of claim 1, wherein the individual is a child having an age of less than 18 years old.

9. The method of claim 8, wherein the age is less than 10 years old.

10. The method of claim 1, wherein the stimulation protocol is sufficient to increase an intelligence, emotional stability, memory retention, alertness and/or speed of learning of the individual.

11. The method of claim 1, wherein the electrical impulse is sufficient to modify a microglia in a central nervous system of the individual.

12. The method of claim 15, wherein the electrical impulse is sufficient to alter the microglia from a substantially pro-inflammatory state to a substantially non-inflammatory state.

13. A method comprising:
applying an electrical impulse to a vagus nerve within the individual according to a stimulation protocol that includes at least two doses administered each day for a plurality of days, wherein the doses each have a duration of about ninety seconds to about 3 minutes; and
wherein the stimulation protocol is sufficient to increase an effectiveness of the neural network in the brain of the individual.

14. The method of claim 13, wherein the stimulation protocol is sufficient to increase a connectivity of neurons within the brain of the individual.

15. The method of claim 13, wherein the stimulation protocol is sufficient to increase a neuronal plasticity within the brain of the individual.

16. The method of claim 13, wherein the doses are separated by a time frame of about five to 15 minutes.

17. The method of claim 13, wherein the stimulation protocol comprises 2 to 12 treatments/day.

18. The method of claim 13 wherein the stimulation protocol is administered every day for at least 90 days.

19. The method of claim 13, wherein the stimulation protocol is administered every day for at least one year.

20. The method of claim 1, wherein the stimulation protocol is sufficient to increase an intelligence, emotional stability, memory retention, alertness and/or speed of learning of the individual.
